(19) United States
(12) Patent Application Publication
(10) Pub. No.: US 2025/0102527 A1
(43) Pub. Date: Mar. 27, 2025

(54) METHOD AND DEVICE FOR DETERMINING THE VITAMIN STATUS

(71) Applicant: TECHNISCHE UNIVERSITÄT MÜNCHEN, MÜNCHEN (DE)

(72) Inventors: Stephan SIEBER, UTTING (DE); Nina BACH, WÖRTH (DE); Michaela FIEDLER, RUHPOLDING (DE); Jonas DRECHSEL, MÜNCHEN (DE)

(21) Appl. No.: 18/834,096

(22) PCT Filed: Apr. 3, 2023
(86) PCT No.: PCT/EP2023/058690

§ 371 Date: Jul. 29, 2024

(30) Foreign Application Priority Data
Apr. 11, 2022 (EP) 22167551.5

(51) Int. Cl. G01N 33/82; G01N 33/543; G01N 33/573; G01N 33/58

(52) U.S. Cl. CPC G01N 33/82; G01N 33/54366; G01N 33/573; G01N 33/582

(57) ABSTRACT

The present invention relates to in vitro methods for determining the vitamin status of a subject, in particular health related vitamins, such as B2, B6, B12 and/or D3. The present invention further relates to a kit and an immunographic device for in vitro determining the vitamin status of a subject.

The present invention relates to in vitro methods for determining the vitamin status of a subject, in particular health related vitamins, such as B2, B6, B12 and/or D3. The present invention further relates to a kit and an immunographic device for in vitro determining the vitamin status of a subject.

### BACKGROUND OF THE INVENTION

Vitamin B6 refers to a collection of vitamers including the enzyme cofactor pyridoxal phosphate (PLP). B6 is an essential nutrient that has to be taken up by a well-balanced diet. While the supply of B6 in wholesome foods including meat, bananas and potatoes is sufficient, processing of meals can significantly reduce the amount of B6 vitamers due to their limited stability (Gregory and Kirk, 1977).

The Centers for Disease Control and Prevention showed in a study that about 10% of the US population exhibits plasma PLP levels which are below 20 nmol/L, considered as threshold value for sufficient supply, indicating an inadequate B6 status (Center for Disease Control and Prevention, 2012). Clinical signs of an apparent vitamin B6 deficiency are neurological disorders (ataxia, paresis), skin and mucous membrane changes, blood cell cytopenia, and increase of plasma homocysteine concentrations. B6 deficiency is also associated with various symptoms including higher rates of cardiovascular disease (arteriosclerosis, coronary heart disease), stroke and cancer (Verhoef et al., 1996; Robinson et al., 1998; Sies et al., 1992; Vanuzzo et al., 2007; Dalery et al., 1995; Rimm et al., 1998; Johansson et al., 2010; Kelly et al., 2004; Lotto et al., 2011). Populations at risk to exhibit critically low B6 levels include elderly people, diabetes patients, oral conceptive users and alcoholics. Malnutrition, as often observed in developing countries, further intensifies the problem. Additionally, healthy people with increased needs of B6, such as pregnant and lactating women require a stringent and continuous monitoring of B6 levels as fetal and infant brain development relies on adequate B6 levels (Driskell, 1994).

Several diagnostic procedures are on the market which are classified by either direct or indirect functional measures. For example, plasma PLP is the most prevalent biomarker used (Leklem, 1990), which is detected by high performance liquid chromatography (HPLC) as part of laboratory diagnostics. Major challenges in its determination are the
overall stability of PLP, whose plasma concentration declines in samples kept at room temperature and exposed to light (Hustad et al., 2012; Panton et al., 2013; van der Ham et al., 2012);
inflammation, albumin binding, alkaline phosphatase activity and alcohol consumption can result in a 30-40% variance (Brussaard et al., 1997; Ueland et al., 2015).

Thus, erythrocyte PLP determination via HPLC or functional assays have been identified as a more relevant marker of the B6 status also reflecting the fact, that the vitamer is an intracellular cofactor (Leklem, 1990; Ueland et al., 2015; Vermaak et al., 1990). Blood can be rapidly drawn from patients providing an opportunity to directly monitor effects of B6 supplementation. However, a disadvantage of direct PLP measurement is the binding of PLP to hemoglobin, lowering the overall precision (Ueland et al., 2015; Reynolds et al., 1995). As an alternative, functional B6 biomarker tests are based on PLP-dependent enzymes (PLP-DEs) that are supposed to overcome these problems. Here, the transaminase test is one of the most mature assay systems monitoring the activity of aspartic acid transaminase or alanine transaminase in erythrocyte extract (Ueland et al., 2015). While this assay overcomes above mentioned limitations of free-PLP detection, a major challenge is the time-consuming processing of fresh blood for these assays limiting the overall stability of PLP-DEs (Ueland et al., 2015; Huang et al., 1998).

Until now, there is no diagnostic B6 test available which combines analysis of blood samples, detection of most relevant enzyme based-PLP-cofactor status and speed for a reliable analysis. No personalized parameters which are based on recording the status of multiple enzymes can be obtained via the methods of the prior art.

There is a need in the art for improved methods and means for determining the vitamin status of a subject.

### SUMMARY OF THE INVENTION

According to the present invention this object is solved by an in vitro method for determining the vitamin status of a subject comprising
(a) harvesting erythrocytes from a sample obtained from a subject or providing erythrocytes obtained from a sample of a subject;
(b) providing a probe comprising at least one functionalized cofactor of PLP-dependent enzymes (PLP-DEs) (PLP probe);
(c) lysing the erythrocytes and thereby releasing the proteome including the PLP-DEs and then treating the lysate with the PLP probe provided in (b); or
(c′) treating the erythrocytes with the PLP probe provided in (b) and then lysing the erythrocytes and thereby releasing the proteome including the PLP-DEs;
(d) adding a label for the PLP-probe to the treated lysate of (c) or to the lysate of (c′);
(e) capturing the labeled PLP-DEs with anti-PLP-DE-antibodies immobilized on a surface, preferably an array or a membrane,
(f) detecting the labeled PLP-DEs, and
(g) determining the vitamin status.

According to the present invention this object is solved by a kit for in vitro the vitamin status, in particular vitamin B6, B2, B12 and/or D3, in a sample of a subject, comprising
(1) a probe comprising at least one functionalized cofactor of PLP-dependent enzymes (PLP-DEs) (PLP probe);
(2) a label for the PLP-probe;
(3) optionally, an array for PLP-DEs, preferably comprising anti-PLP-DE-antibodies,
(4) optionally, reagents for lysing erythrocytes, reducing agents, and control(s).

According to the present invention this object is solved by an immunographic device for in vitro determining the vitamin status, in particular vitamin B6, B2, B12 and/or D3, in a sample of a subject,
said device comprising a solid carrier or surface coated with anti-PLP-DE-antibodies, wherein the immunographic device is preferably a lateral flow assay (LFA) device.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to 21” should be interpreted to include not only the explicitly recited values of 1 to 21, but also include individual values and sub-ranges within the indicated range. Thus, included in this numerical range are individual values such as 1, 2, 3, 4, 5 . . . 17, 18, 19, 20, 21 and sub-ranges such as from 2 to 10, 8 to 15, etc. This same principle applies to ranges reciting only one numerical value, such as “at least 90%”. Furthermore, such an interpretation should apply regardless of the breadth of the range or the characteristics being described.

### Method for Determining the Vitamin Status

As outlined above, the present invention provides an in vitro method for determining the vitamin status of a subject.

Preferably, the vitamin status of health related vitamins is determined, in particular vitamin B2, B6, B12 and/or D3. In a preferred embodiment, the vitamin B6 status is determined.

Said method comprises the steps of:
(a) harvesting erythrocytes from a sample obtained from a subject or providing erythrocytes obtained from a sample of a subject;
(b) providing a probe comprising at least one functionalized cofactor of PLP-dependent enzymes (PLP-DEs) (PLP probe);
(c) lysing the erythrocytes and thereby releasing the proteome including the PLP-DEs and then treating the lysate with the PLP probe provided in (b);
or
(c′) treating the erythrocytes with the PLP probe provided in (b) and then lysing the erythrocytes and thereby releasing the proteome including the PLP-DEs;
(d) adding a label for the PLP-probe to the treated lysate of (c) or to the lysate of (c′);
(e) capturing the labeled PLP-DEs with anti-PLP-DE-antibodies immobilized on a surface, preferably an array or a membrane,
(f) detecting the labeled PLP-DEs, and
(g) determining the vitamin status.

In one embodiment, the method comprises the steps of:
(a) harvesting erythrocytes from a sample obtained from a subject or providing erythrocytes obtained from a sample of a subject;
(b) providing a probe comprising at least one functionalized cofactor of PLP-dependent enzymes (PLP-DEs) (PLP probe);
(c) lysing the erythrocytes and thereby releasing the proteome including the PLP-DEs and then treating the lysate with the PLP probe provided in (b);
(d) adding a label for the PLP-probe to the treated lysate of (c);
(e) capturing the labeled PLP-DEs with anti-PLP-DE-antibodies immobilized on a surface, preferably an array or a membrane,
(f) detecting the labeled PLP-DEs, and
(g) determining the vitamin status.

In an alternative embodiment, the method comprises the steps of:
(a) harvesting erythrocytes from a sample obtained from a subject or providing erythrocytes obtained from a sample of a subject;
(b) providing a probe comprising at least one functionalized cofactor of PLP-dependent enzymes (PLP-DEs) (PLP probe);
(c′) treating the erythrocytes with the PLP probe provided in (b) and then lysing the erythrocytes and thereby releasing the proteome including the PLP-DEs;
(d) adding a label for the PLP-probe to the lysate of (c′);
(e) capturing the labeled PLP-DEs with anti-PLP-DE-antibodies immobilized on a surface, preferably an array or a membrane,
(f) detecting the labeled PLP-DEs, and
(g) determining the vitamin status.

### Step (a)

In step (a) of the method of the present invention, erythrocytes are harvested from a sample obtained from a subject or erythrocytes obtained from a sample of a subject are provided.

In the embodiment, where the erythrocytes are harvested from a sample, they are preferably harvested by centrifugation, optionally including washing step(s).

The sample is preferably whole blood.

### Step (b)

In step (b) of the method of the present invention, a probe is provided which comprises at least one functionalized cofactor of PLP-dependent enzymes (PLP-DEs) (PLP probe).

PLP-DEs have been implicated in human disease and are recognized as important drug targets. PLP-DEs include aspartic acid transaminase, alanine racemase, serine hydroxymethyltransferase, ornithine decarboxylase, GABA aminotransferase, DOPA decarboxylase, and branched-chain amino acid aminotransferase. PLP acts as a coenzyme in all transamination reactions, and in certain decarboxylation, deamination, and racemization reactions of amino acids. PLP is also involved in various beta-elimination reactions such as the reactions carried out by serine dehydratase and GDP-4-keto-6-deoxymannose-3-dehydratase (Co1D).

Preferably, the cofactor(s) of PLP-dependent enzymes (PLP-DEs) are functionalized with a biorthogonal labeling group.

In one embodiment, the cofactor(s) of PLP-dependent enzymes (PLP-DEs) are functionalized with a biorthogonal labeling group and optionally further with a phosphoramidate capping group.

The term “bioorthogonal chemistry” refers to any chemical reactions that can occur inside of living systems or in biological samples in general, that do not interfere with the biological system as well as side reactions in the organism or with the biological sample do not take place making bioorthogonal chemistry extremely selective reactions within complex biological matrices.

A number of chemical ligation strategies have been developed that fulfill the requirements of bioorthogonality, including
1,3-dipolar cycloaddition between azides and cyclooctynes (also termed copper-free click chemistry), between nitrones and cyclooctynes, oxime/hydrazone formation from aldehydes and ketones, the tetrazine ligation, the isocyanide-based click reaction, and the quadricyclane ligation. Furthermore the copper catalyzed azide alkyne click reaction (CuAAC) and the Staudinger ligation.

The use of bioorthogonal chemistry usually proceeds in two steps. First, a (cellular) substrate is modified with a bioorthogonal functional group (“biorthogonal labeling group”). Secondly, a probe containing the complementary functional group is introduced to react and label the substrate.

In a preferred embodiment, the biorthogonal labeling group is used to modify or functionalize the cofactor(s) of the PLP-DEs. The complementary functional group is added to the label.

The biorthogonal labeling group is preferably selected from an alkyne or azide moiety or a tetrazine functionality.

In a preferred embodiment, the biorthogonal labeling group is an alkyne group. The alkyne group can be attached directly to the pyridine ring of the cofactor (such as PL1 or PL1P) or with a spacer, such as an ethylene spacer (such as PL2 or PL2P).

The phosphorylated probes (PLxP) can be obtained from the reaction of the respective PLx probe with the enzyme S. Aureus protein kinase PLK, such as described in Hoegl et al. (2018) or Fux et al. (2019).

The functionalized cofactors are preferably selected from PL1P, PL2P, PL4P to PL13P.
PL1P: (6-ethynyl-4-formyl-5-hydroxypyridin-3-yl)methyl phosphate
PL2P: (6-(but-3-yn-1-yl)-4-formyl-5-hydroxypyridin-3-yl)methyl phosphate
PL4P: (4-formyl-6-(hept-6-yn-1-yl)-5-hydroxypyridin-3-yl)methyl phosphate
PL5P: (6-(4-ethynylphenethyl)-4-formyl-5-hydroxypyridin-3-yl)methyl phosphate
PL6P: (4-formyl-5-hydroxy-6-(pent-4-yn-1-yl) pyridin-3-yl)methyl phosphate
PL7P: (4-formyl-6-(hex-5-yn-1-yl)-5-hydroxypyridin-3-yl)methyl phosphate
PL8P: (4-formyl-5-hydroxy-6-(2-(1-(prop-2-yn-1-yl)-1H-1,2,3-triazol-4-yl)ethyl) pyridin-3-yl)methyl phosphate
PL9P: (6-(2-(1-(but-3-yn-1-yl)-1H-1,2,3-triazol-4-yl)ethyl)-4-formyl-5-hydroxypyridin-3-yl)methyl phosphate
PL10P: (2-ethynyl-4-formyl-5-hydroxy-6-methylpyridin-3-yl)methyl phosphate
PL11P: (4-formyl-5-hydroxy-6-methyl-2-(1-(prop-2-yn-1-yl)-1H-1,2,3-triazol-4-yl) pyridin-3-yl)methyl phosphate
PL12P: (2-(but-3-yn-1-yl)-4-formyl-5-hydroxy-6-methylpyridin-3-yl)methyl phosphate
PL13P: (4-formyl-6-methyl-5-(prop-2-yn-1-yloxy)pyridin-3-yl)methyl phosphate,

In a preferred embodiment, the biorthogonal labeling group is an azide group, and the cofactor is preferably PL3P.
PL3P: (6-(azidomethyl)-4-formyl-5-hydroxypyridin-3-yl)methyl phosphate

In a preferred embodiment, the cofactor of PLP-DEs is further functionalized with a phosphoramidate.

In one embodiment, the probe comprises at least two functionalized cofactors of PLP-DEs or it comprises three or more functionalized cofactors.

In one embodiment, the probe comprises a further moiety or tag or further moieties/tags, such as biotin.

### Step (c)

In step (c) of the method of the present invention, the erythrocytes are lysed and thereby release the proteome including the PLP-DEs.

Preferably, the lysis of the erythrocytes comprises the removal of hemoglobin. Removal of hemoglobin is preferred, because PLP binds to hemoglobin, which can disturb the result of the method of the present invention.

In one embodiment, step (c) comprises the addition of a lysis buffer, preferably in a ratio erythrocytes:buffer=1:1. The lysis buffer can be any suitable buffer to lyse the cells, such as Tris buffer (Tris 50 mM, NaCl 150 mM, 1% NP40 (v/v), pH=7.5). Step (c) can further comprise incubation with lysis buffer (preferably on ice) for 5 min and removal of hemoglobin with NiNTA-agarose beads (e.g. ratio˜10 μL lysate to 100 μL beads).

Step (c) of the method of the present invention further comprises that the lysate obtained is treated with the PLP probe provided in (b).

Preferably, the treatment comprises an incubation of the lysate with the PLP probe and optional addition of a reducing agent.

Under reducing conditions, the PLP probe will bind irreversibly to the PLP DEs.

In one embodiment, reduction can be performed—if necessary—with sodium borohydride NaBH₄.

Step (c′)

In step (c′) of the method of the present invention, the erythrocytes are treated with the PLP probe provided in (b).

Said treatment with the PLP probe is carried out before lysing the erythrocytes.

Preferably, the PLP probe is a cofactor of PLP-DEs which is (further) functionalized with a phosphoramidate.

Preferably, the treatment comprises an incubation of the lysate with the PLP probe and optional addition of a reducing agent.

In step (c′), after the treatment, the erythrocytes are lysed and thereby release the proteome including the PLP-DEs.

Preferably, the lysis of the erythrocytes comprises the removal of hemoglobin. Removal of hemoglobin is preferred, because PLP binds to hemoglobin, which can disturb the result of the method of the present invention.

In one embodiment, step (c′) comprises the addition of a lysis buffer, preferably in a ratio erythrocytes:buffer=1:1. The lysis buffer can be any suitable buffer to lyse the cells, such as Tris buffer (Tris 50 mM, NaCl 150 mM, 1% NP40 (v/v), pH=7.5). Step (c) can further comprise incubation with lysis buffer (preferably on ice) for 5 min and removal of hemoglobin with NiNTA-agarose beads (e.g. ratio˜10 μL lysate to 100 μL beads).

### Step (d)

In step (d) of the method of the present invention, a label for the PLP-probe is added to the treated lysate of (c) or the lysate of (c′).

Preferably, the label in step (d) is
a fluorescent label,
   such as rhodamines, cyanine dyes (Cy3, Cy5), fluoresceines, a bioluminescence or chemiluminescence label,
   such as isoluminol and its derivatives, luciferins,
a dye,
or combinations thereof.

The label is preferably attached to the biorthogonal labeling group of the cofactor(s). The label preferably comprises a complementary functional group to the biorthogonal labeling group of the cofactor(s). Both the biorthogonal labeling group and the respective complementary functional group react with each other, such as via click chemistry or Staudinger ligation.

### Step (e)

In step (e) of the method of the present invention, the labeled PLP-DEs are captured with anti-PLP-DE-antibodies immobilized on a surface.

The surface is preferably an array or a membrane. Suitable surfaces are known to the skilled artisan.

The anti-PLP-DE-antibodies can be monoclonal or polyclonal antibodies. Such antibodies are commercially available. For example:
Anti PLPHP_1 Thermofischer, cat. nr. PA532036, polyclonal
Anti PLPBP_2 Novus Biologicals, cat. No. 21730002 polyclonal
Anti PLPBP_3 Origene, cat. nr. TA505130, monoclonal
Anti OAT_1 Origene, cat. AM09363PU-S, polyclonal
Anti OAT_2 Abcam, cat. Ab137679, polyclonal
Anti OAT_3 Novus Biologicals, cat. NBP1-49312, monoclonal
Anti SHMT1_1 Abcam, cat. Ab186130, polyclonal
Anti SHMT1_2 Merck, cat. ABE416, polyclonal
Anti SHMT1_3 Novus Biologicals, cat. NBP2-32173, polyclonal

In a preferred embodiment, in or after step (e) a second anti-PLP-DE-antibody is added.

Said second anti-PLP-DE-antibody is not immobilized on a surface. Said second anti-PLP-DE-antibody comprises a label, which is different from the label of the PLxP-probe.

Preferably, the label of the second anti-PLP-DE-antibody is
a fluorescent label,
   such as rhodamines, cyanine dyes (Cy3, Cy5), fluoresceines, a bioluminescence or chemiluminescence label,
   such as isoluminol and its derivatives, luciferins,
a dye,
or combinations thereof.

For example, the second anti-PLP-DE-antibody is a polyclonal antibody which can be the same as the capturing anti-PLP-DE-antibody due to its polyclonality.

### Step (f)

In step (f) of the method of the present invention, the labeled PLP-DEs are detected.

The method of detection and the readout depend on the label used.

In an embodiment, where the label is a fluorescent label, the fluorescence is detected and preferably fluorescence intensity is determined. The fluorescence intensity can be the readout. In an embodiment, where the label is a bioluminescence label, the bioluminescence is detected and preferably bioluminescence intensity is determined. The bioluminescence intensity can be the readout.

In an embodiment, where the label is a dye, the color can be detected or absorbance can be detected.

In an embodiment, where second anti-PLP-DE-antibodies are used, the label of said second antibody is detected as well.

In this embodiment, two signals are detected, where (1) the PLP-probe signal reflects the loading state of the cofactor (wherein higher PLP probe signal corresponds to a lower loading state of the cofactor) and (2) the antibody signal reflects the total amount of PLP-DE enzyme in the sample. By comparing these to signals, a ratio PLP loading state/total PLP-DE amount is obtained.

Healthy patients exhibit roughly the same ratio, whereas vitamin B6 compromised patients exhibit higher ratios.

For example, in case of vitamin B6 deficiency, little PLP is bound to PLP-DE, such that more PLxP probe can bind to the PLP-DEs which results in a higher signal.

Additionally, the free vitamin B6 (free PLP) status of the patients in blood can be determined via standard HPLC methods to calibrate the ratios.

### Step (g)

In step (g) of the method of the present invention, the vitamin status is determined.

Preferably, the vitamin status is determined by comparing the readout of step (f), such as fluorescence intensity, bioluminescence intensity, or absorbance, with a control. Preferred controls are recombinant PLP-DEs with defined cofactor loading or reference samples where the PLP status is known, e.g. in form of a calibration curve.

Preferably, the vitamin B6 status of the subject is determined.

In a preferred embodiment, the method of the present invention is a point of care method.

Point-of-care testing (POCT) is a form of testing in which the analysis is performed where healthcare is provided close to or near the patient. POCT can be undertaken in many locations including: home use and self testing.

In preferred embodiments, the method of the present invention further comprises the steps of:
dividing the sample obtained from the subject in two parts and pre-treating one part with a PLP standard prior to step (c),
performing steps (a) to (f) for both parts in parallel,
comparing the results of step (f) for both parts and thereby quantifying the PLP status.

Preferably, the vitamin status determined in a subject is used for determining the vitamin B6 status of the subject.

Preferably, the vitamin status determined in a subject is used for monitoring the PLP status and/or vitamin B6 status of the subject.

Preferably, the vitamin status determined in a subject is used for diagnosing vitamin B6-deficiency and vitamin B6-deficiency-associated diseases,
in particular related to age, malnutrition, and/or alcoholism.
in dialysis patients,
inflammatory bowel disease.

Vitamin B6 comprises six compounds based on a 2-methyl-3-hydroxypyridine structure with differing subunits at positions C4 and C5 that are interconvertible. These are pyridoxine, pyridoxamine, and pyridoxal and their phosphorylated derivatives pyridoxine-5-phosphate, pyridoxamine-5-phosphate, and pyridoxal-5-phosphate (PLP). PLP is a cofactor in >150 different enzyme reactions including transamination and decarboxylation enzyme systems. The symptoms of vitamin B6 deficiency include peripheral neuropathy, pellagra-like syndrome with seborrheic dermatitis and glossitis. Prolonged deficiency can lead to depression, confusion, and in severe cases causes abnormalities in EEG signals and seizures. Vitamin B6 deficiency is often associated with deficiencies in other B vitamins and can be due to overall poor nutrition caused by a chaotic lifestyle, e.g., in alcoholic subjects. Suboptimal vitamin B6 status has also been reported in oral contraceptive users, smokers, and patients with celiac disease or diabetes Secondary vitamin B6 deficiency can occur in treatments with drugs that interact with PLP, e.g., isoniazid or inborn errors in vitamin B6 salvage pathways or when mutations result in the accumulation of intermediates that react with PLP. Vitamin B6 deficiency has been reported to be associated with an increased risk of cardiovascular disease, stroke, and cancer. Low plasma PLP has been reported in a number of diseases associated with inflammation including rheumatoid arthritis, inflammatory bowel disease, diabetes, and deep vein thrombosis.

### Test Device and Kit

As outlined above, the present invention provides a kit for in vitro determining the vitamin status in a sample of a subject.

Preferably, the vitamin status of health related vitamins is determined, in particular vitamin B2, B6, B12 and/or D3. In a preferred embodiment, the vitamin B6 status is determined.

Said kit comprises
(1) a probe comprising at least one functionalized cofactor of PLP-dependent enzymes (PLP-DEs) (PLP probe);
(2) a label for the PLP-probe;
(3) optionally, an array for PLP-DEs, preferably comprising anti-PLP-DE-antibodies,
(4) optionally, reagents for lysing erythrocytes, reducing agents, and control(s).

As outlined above, the present invention provides an immunographic device for in vitro determining the vitamin status in a sample of a subject.

Preferably, the probe comprising at least one functionalized cofactor of PLP-dependent enzymes (PLP-DEs) (PLP probe) is as defined herein above.

Preferably, the label for the PLP-probe is as defined herein above.

Preferably, the vitamin status of health related vitamins is determined, in particular vitamin B2, B6, B12 and/or D3. In a preferred embodiment, the vitamin B6 status is determined.

Said device comprises a solid carrier or surface coated with anti-PLP-DE-antibodies or a solid carrier or surface coated with nanoparticles comprising a moiety or tag which binds to a probe comprising at least one functionalized cofactor of PLP-dependent enzymes (PLP-DEs) (PLP probe).

Preferably, the immunographic device is a lateral flow assay (LFA) device, which is more preferably a point-of-care device.

In an embodiment, the immunographic device comprises a porous membrane operably connected to
(a) a portion or pad, where a processed sample is applied,
(b) a test portion or test line comprising said anti-PLP-DE-antibodies or said nanoparticles, preferably gold nanoparticles,
(c) a control portion or test line; and
(d) an absorbent portion/pad.

Preferably, the sample is whole blood or erythrocytes.

In an embodiment, when the sample is whole blood, the erythrocytes are harvested therefrom, as described above

In a preferred embodiment, obtaining a processed sample, which is applied to the device of the present invention, comprises:
the erythrocytes or the harvested erythrocytes are lysed and thereby release the proteome including the PLP-dependent enzymes (PLP-DEs),
the lysate is treated with a probe comprising at least one functionalized cofactor of PLP-dependent enzymes (PLP-DEs) (PLP probe), said probe being as defined herein above, and
a label for the PLP-probe, which is as defined herein above, is added to the treated lysate, and
the processed sample is obtained.

In an alternative embodiment, obtaining a processed sample, which is applied to the device of the present invention, comprises:
the erythrocytes or the harvested erythrocytes are treated with a probe comprising at least one functionalized cofactor of PLP-dependent enzymes (PLP-DEs) (PLP probe), said probe being preferably a cofactor of PLP-DEs which is functionalized with a phosphoramidate,
the treated erythrocytes are then lysed and thereby release the proteome including the PLP-dependent enzymes (PLP-DEs),
a label for the PLP-probe, which is as defined herein above, is added to the lysate, and
the processed sample is obtained.

In an embodiment, the PLP-probe comprises a biotin tag and the gold nanoparticles in the test portion/line are streptavidin-gold nanoparticles.

Preferably, the control portion or control line comprises antibodies against streptavidin, or biotin or biotin derivatives to capture the streptavidin-gold nanoparticles.

### PREFERRED EMBODIMENTS

The inventors developed chemical PLP probes which successfully captured 75% of all known bacterial PLP-DEs. These probes largely mimic the pyridoxal core scaffold and bear a biorthogonal alkyne handle for click chemistry to a reporter tag. In a previous study, published under Hoegl A. et al. (Nature Chemistry, 2018), we showed that the probes infiltrate cognate pyridoxal uptake into bacteria, get phosphorylated to the corresponding PLP probes by pyridoxal kinases and finally become incorporated into PLP-DEs. The inventors also applied this methodology to study the human PLP-ome (published under Fux et al. (Cell Chemical Biology, 2019), where they demonstrated the utility of these PLP-cofactor mimics for the identification of human PLP-binding proteins in live cells.

In the present application, it is first shown that PLP probes are highly versatile tools to directly determine the PLP status in human erythrocytes. See FIG. 2.

Human erythrocytes express seven known PLP-dependent enzymes (Bryk et al., 2017) and the activity of PLP-DEs is generally dependent on the amount of PLP bound (Ueland et al., 2015). The inventors show that a PLP probe can immediately monitor the PLP bound status of all seven enzymes in parallel without the need of performing individual activity assays. This not only enhances the reliability but also provides personalized parameters due to an assessment of multiple enzymes with essential cellular functions. For example, if the PLP status is low more probe binding will be observed to these enzymes and accordingly a higher probe-bound PLP-DE signal will be obtained. If the levels are high, the signal goes down. Such a direct assay provides a readout of the overall B6 status (direct parameter) and furthermore yields individual parameters on several signature PLP-DEs which may correlate with individual disease predispositions (personalized parameter).

The invention provides a method as well as a ready-to-use device, termed B6VitaStat, which is able to directly monitor the PLP status of human erythrocytes based on multiple readout parameters. Erythrocytes are treated with our PLP cofactor mimics. Upon lysis of erythrocytes, PLP-DEs are labeled with our PLP probe and subsequently clicked to a fluorescent rhodamine tag. Labeled proteins are captured by commercial antibodies against PLP-DEs which are immobilized on a surface, such as glass slides (microarray) or membranes. The array is washed and analyzed in a fluorescent scanner. Recombinant PLP-DEs with defined cofactor loading are added to the lysate as internal standard to provide a quality control and facilitate a quantitative readout by comparison of signal intensities (FIG. 3).

Alternatively for relative quantification, the patient's blood is split into two equivalent samples and one is pre-incubated with a defined PLP standard prior to probe treatment. Thus, less PLP probe can bind to the human PLP-DEs resulting in a lower (fluorescent) signal, whereas more probe is able to bind the non-pretreated sample leading to higher signals. Comparison of the obtained signals upon (fluorescent) readout on the microarray allows the determination of the patient's PLP status. Implementation of threshold values allows to facilitate the recognition of PLP deficiencies in humans (FIG. 4).

Thus, the method of the present invention is able to determine the PLP status immediately, preserves the facile cofactor prior to degradation, provides personalized parameters based on multiple enzymes and is independent from unbound PLP in erythrocytes. Most importantly, this methodology does not require expensive HPLC-instruments and only a much cheaper fluorescent device is needed. Thus, the corresponding test can also be performed easily in developing countries, where malnutrition and B6 deficiency is a big problem.

Signal detection and amplification can also be adjusted to different settings including smartphone-based sensing methods (Kanchi et al., 2018) paving the way for a ready-to-use device at family doctor offices or at home.

Our novel B6VitaStat platform is innovative as it provides a new approach to overcome limitations of established methods suffering from long processing times, indirect readouts, narrow scope and need for high-end instruments. Currently, tests comprise direct PLP determination via HPLC analysis or indirect PLP determination based on enzymatic activity (such as disclosed in German patent No. DE 000060037311 T2) or bioactivity (e.g. ID-Vit®-Vitamin B6 Assay from Immundiagnostik, Germany). In contrast, B6VitaStat according to the present invention represents an improved diagnostic platform for continuous and direct monitoring of the vitamin B6 status directly from patient blood without the need of expensive equipment providing rapid processing analysis. This kind of monitoring is easy and can also be performed by nonmedical experts, e.g. at home or in developing countries. Like Vitamin D which can be detected by different immunoassay methods, such as electrochemiluminescent assay Elecsys® (Roche Diagnostics) or Preventis SmarTest Pro® Vitamin D, B6 determination can now be offered on a more frequent basis or even independent of diagnostic laboratories. For example, comparison of blood samples from different patients suffering under diverse B6-deficiency associated diseases will likely provide patterns characteristic for the respective pathologies. B6VitaStat thus not only provides rapid and cheap access to the nutritional B6 status but can also serve as a device linked to machine learning with important implications for personalized medicine and diagnostics.

Furthermore, due to the dependency of the fluorescence intensity of the amount of protein present in the sample and the saturation state of the enzyme (i.e. the PLP loading status of PLP-DEs), the microarray can be extended to a sandwich format to enable normalization of signal intensities and to interpret the vitamin status of a subject. Therefore, after incubation of the PLP treated lysate on the microarrays, a second antibody bearing a different fluorescent label than the PLP probe is added and a two-channel fluorescence readout is performed. Herein, high ratios of PLP probe/protein amount indicate low vitamin B6 status. Comparison of two healthy volunteers with differences in their vitamin B6 status (as pre-determined via HPLC) verified this theory making this platform a novel platform for vitamin status assessment (see e.g. FIG. 12).

### Conclusion

Monitoring health and disease via self-diagnostic point of care devices is a growing field with established examples for blood sugar determination in diabetes patients. However, vitamin deficiencies due to malnutrition in developing countries and unhealthy lifestyles including less-balanced diets in the western world are of growing concern. The consolidated detection of these vitamins is still laborious and complicated due to a lack of suitable methods.

We here demonstrate that the direct readout of vitamin B6 binding to PLP-DE active sites via chemical probes enables a fast and reliable method for a qualitative vitamin status assessment. The assay disclosed herein requires probe addition to hemoglobin depleted erythrocyte lysate, Click reaction and subsequent analysis on an array. The array operates with a suitable dynamic range for covering relevant B6 concentrations and the results are in line with corresponding HPLC benchmark analyses.

In addition to the fast and easy handling, our method does not rely on the transient stability of free vitamins as well as fluctuations of levels e.g. due to nutritional uptake, but rather monitors their long-term levels in protein-bound forms. We demonstrate that the device operates well with several independent PLP-DEs thus enhancing the overall fidelity. Vitamin B6 is one of multiple vitamins amenable to this technology. Their consolidated readout via our probe technology provides the basis for a better and real time monitoring of health and disease at home or in places lacking a functional medical infrastructure.

The following examples and drawings illustrate the present invention without, however, limiting the same thereto.

### BRIEF DESCRIPTION OF THE DRAWINGS

FIG. 1. PLP probes.
A, Chemical structures of phosphorylated PLP probes (PLxP).
B, Exemplary structure of PLP probes with a further phosphoramidate group.

FIG. 2. Labeling strategy to detect PLP-dependent enzymes with PLP probes.

Desired PLP probe is incubated with human erythrocyte lysate, incorporated into human PLP-DEs (big crescent moon=enzyme). Introduction of a fluorescent tag via click chemistry allows visualization of PLP-DEs.

FIG. 3. Schematic workflow of the method of the present invention.

Erythrocytes are isolated from patient blood samples (1), lysed (2) and retrieved proteome is incubated with PLxP probe (3). Via Click chemistry a fluorescent dye is attached to the complex of probe and PLP-DE protein (4). The whole complex is captured with antibodies against defined PLP-DE proteins, which are imprinted on microarray glass slides (5) enabling the fluorescent signal readout of bound probe on microarray format (6). Fluorescence readout allows direct determination of the patient's PLP status.

FIG. 4. Method of the present invention using second anti-PLP-DE antibodies.

The PLP-DE enzymes in the lysate are labelled with the PLP cofactor probes and bioorthogonally attached to a label. After capturing these labelled PLP-DE enzymes, a second polyclonal antibody (that can be the same as the capturing antibody due to its polyclonality) labelled with a second label different to the PLP-probe label. Two different monoclonal antibodies on the surface and as a second labeled antibody are also possible. Two signals can be detected, where the PLP-probe signal reflects the loading state of the cofactor and the antibody signal reflects the total amount of PLP-DE enzyme in the sample. By comparing these two signals, a ratio PLP loading state/total PLP-DE amount can be obtained. High ratios indicate low endogenous PLP loading states and vice versa.

FIG. 5. Schematic workflow of the method of the present invention for relative quantification of PLP status.

Interpretation of received microarray signals: High fluorescent signal intensities indicate a high level of bound PLxP probe and are inversely correlated with a low endogenous PLP saturation (bottom). Vice versa low signal intensities point to sufficient levels of endogenous protein-bound PLP (top).

FIG. 6. Recombinant labelling of human PLPBP with different PLP cofactor mimics PL1P, PL2P and PL13P.

Conditions: 1 μM PLPBP, 100 μM probe (incubation 1 h, r.t.), reduction (NaBH₄), precipitation (Aceton), wash (MeOH), redissolve in PBS+0.2% SDS, click to Cy5. Analysis via fluorescence SDS-Page.

Structures of the different PLP probes PL1P, PL2P and PL13P (top) that were tested for their labeling efficiency of recombinant PLP binding protein (PLPBP), separately and all three in combination on a fluorescent SDS gel (bottom). The denaturation of PLPBP via increased temperature (heat control, h.c.) prior to labeling with the combination of probes served as negative control. FM=fluorescent marker.

FIG. 7. anti-PLPBP binding of purified PLPBP protein.

Mean signal intensities (n=16) and error bars (SD) after fluorescent readout of the microarray with recombinant PLPBP samples. Sample preparation: 6 μM PLPBP (40 μg/sample), 100 μM PL2P 1 h r.t., precipitation (acetone), wash, redissolve in PBS, Click to Cy5 1 h r.t., precipitation (acetone), wash, redissolve 2% Tween20. Cy5-PLPBP: 6 μM PLPBP, +BSA: 6 μM PLPBP (40 μg)+bovine serum albumin (40 μg), +Plasma (40 μg): 6 μM PLPBP+plasma (40 μg); empty ctrl:background signal (no antibody spotted), BSA ctrl:background signal with BSA on array spotted (measure for unspecific binding). Experiment performed in duplicates (n_{bio}=2).

FIG. 8. Workflow optimization via anti-PLPBP binding of purified PLPBP protein.

Signal intensities after fluorescent readout of the microarray with recombinant PLPBP samples. Facilitated sample preparation: (1): 6 μM PLPBP (210 μL→˜40 μg/sample), 100 μM PL2P, 1 h, r.t, Click to Rh—N₃, (2): control sample without PL2P, (3): control sample according to initial sample preparation, but click to Rh—N₃ instead of Cy5.

FIG. 9. Detection of PLP homeostasis protein in erythrocyte lysates (EL).

Mean signal intensities of anti-PLPBPs (n=16) corrected to empty controls and error bars (SD) after fluorescent readout of the microarray with erythrocyte lysate samples without Ni-NTA depletion. Sample preparation/conditions: 0.5 μL erythrocyte lysate in 100 μL PBS, 100 μM PL2P, 1 h, r.t., Click to Rh—N₃. EL spike in samples were prepared equally except for adding 2.0 or 18.0 μg recombinant PLPBP as control prior to sample preparation.

FIG. 10. Example image of a microarray.
(A) Example of obtained image of a microarray after incubation of sample and scanning of fluorescence intensity of the PLP-Probe signal in the Cy3 channel (549 nm). Left and right image represent the same experiment. Experiment shows the signals obtained for erythrocyte lysate (EL) spiked with recombinant PLPBP. On this array 7 different antibodies targeting the PLP-DEs PLPBP, SHMT1 and OAT are immobilized in replicates. Different circles show the positions of the different antibodies on the microarray. The intense signals (lower right and left corner (3 signals) and upper left (2 signals) that are not marked with circles are positional controls to ensure a correct read-out.
(B) Read out of the microarray scan shown in (A) and EL sample without PLPBP spike-in. Mean signal intensities (n=8) corrected to the ctrl empty and error bars (SD) after fluorescent readout of the microarray with erythrocyte lysate. Ctrl=control, EL=erythrocyte lysate, AB=antibody.

FIG. 11. Signal intensity is dependent on saturation level of PLP-DE with PLP.
(A) PLP loading states of recombinant SHMT1 incubated with increasing equivalents (eq) of PLP were determined by intact protein-MS (n=3) as described previously (Hoegl et al., 2018).
(B) Microarray read out of recombinant SHMT1 bound PL2P probe signals preincubated with PLP equivalents in a physiological range of 0.5-4.0 eq. Mean signal intensities of anti-SHMT1 (n=8) corrected to BSA control and error bars (SD) after fluorescent readout of anti-SHMT1 3. Sample preparation according to workflow 3, as described in the Examples.

FIG. 12. Vitamin status assessment-Comparison via PLPBP sandwich readout.

Comparison of two healthy volunteers with differences in their vitamin B6 status (as pre-determined via HPLC) verify the suitability of the method of the present application for vitamin status assessment.
(A) Two-channel fluorescence readout of imprinted anti-PLPBP 2 slides in erythrocyte lysate of two healthy volunteers using PL2P probe clicked to Rh—N₃ and scioDye2 (Sciomics, Germany) labeled polyclonal PLPBP antibody. Mean signal intensities (n=8) corrected to ctrl empty and error bars (SD).
(B) Comparison of microarray read out of signal ratios PL2P/total PLPBP to conventional vitamin B6 values determined by HPLC in medical laboratory. Blood samples of two healthy volunteers with different vitamin B6 status (Person 1: CHPLC (B6)=28 ng/mL, Person 2: CHPLC (B6)=14 ng/ml) roughly reflecting edges of normal vitamin B6 reference range were analysed. Values clearly confirm the inverse correlation of high microarray signal with low HPLC-detected vitamin B6 level and vice versa.

FIG. 13. SDS-Page gel after hemoglobin depletion with Ni-NTA beads in erythrocytes lysate: Conditions: 1) 7 μL EL in 50 μL PBS (−) or 100 μl Ni-NTA bead (+), 30 min, r.t. 2) 7 EL μL lysate in 100 μL PBS (−) or 200 μL Ni-NTA beads (+), 3) 14 μL EL in 100 μL PBS (−) or 200 μL Ni-NTA beads, 30 min, r.t., 4) 14 μL EL in 100 μL PBS (+) or 2×100 μL Ni-NTA beads, 30 min, r.t. RM=Roti® marker. Ni-NTA beads consist of 50% PBS and 50% Ni-NTA beads.

### EXAMPLES

### Example 1 Materials and Methods

1.1 Antibodies
Commercially available antibodies were purchased from
Thermofischer, Novus Biologicals, Origene, Abcam, or
Merck and used upon further purification and stored as indicated.

| Antibody | Company | cat. no. | Clonality |
|---|---|---|---|
| anti-PLPBP_1 | Thermofischer | PA532036 | polyclonal |
| anti-PLPBP_2 | Novus Biologicals | 21730002 | polyclonal |
| anti-PLPBP_3 | Origene | TA505130 | monoclonal |
| anti-OAT_1 | Origene | AM09363PU-S | polyclonal |
| anti-OAT_2 | Abcam | Ab137679 | polyclonal |
| anti-OAT_3 | Novus Biologicals | NBP1-49312 | monoclonal |
| anti-SHMT1_1 | Abcam | Ab186130 | polyclonal |
| anti-SHMT1_2 | Merck | ABE416 | polyclonal |
| anti-SHMT1_3 | Novus Biologicals | NBP2-32173 | polyclonal |

### 1.2 Probe Synthesis

Probes were synthesized and phosphorylation of probes was performed by S. aureus pyridoxal kinase (SaPLK) according to the published procedures in Hoegl et al. (2018) and Pfanzelt et al. (2022).

For example, phoshorylation of PL2 was adapted from in Hoegl et al. (2018) and Pfanzelt et al. (2022). In brief, in vitro phosphorylation of PL-probes using SaPLK: To 400 μL kinase buffer (50 mM Tris, 50 mM KCl, 10 mM MgCl₂, pH=8.0) and 400 μL NP buffer (50 mM NaH₂PO₄, 300 mM NaCl, pH=8.0) were added 10 mM ATP (100 μL of 100 mM stock in ddH₂O, 1.8 mM PL2 (20 μL of 100 mM stock in DMSO) and 67 μM SaPLK (200 μL of 375 μM stock) and incubated overnight at 4° C. with gentle shaking. The solution was filtered through a 10 kDa MWCO centrifugal filter (Sartorius Stedim Biotech) twice to remove SaPLK, and phosphorylated PL2P (1.8 mM) was used in subsequent experiments without further purification, assuming full conversion.

Protein Overexpression of S. aureus PLK was performed as previously described in Hoegl et al. (2018). In brief, E. coli BL21 (DE3) carrying the expression plasmid was cultured in LB-media containing 0.1 mg/ml Ampicillin at 37° C. to an OD₆₀₀=0.6 and expression was induced by adding 1 mM isopropyl-1-thio-β-galactopyranoside (IPTG) for 2 h at 37° C. Bacteria were harvested and washed with PBS (6,000× g, 4° C.). The cell pellet was resuspended in Strep binding buffer (50 mM NaH₂PO₄, 300 mM NaCl, pH=8.0) and lysed by sonication (Bandelin Sonopuls HD 2070, 3× cycles of 70% intensity, 3 min and 30%, 7 min). The lysate was clarified by centrifugation (36,000×g, 30 min, 4° C.). Supernatant was loaded onto a StrepTrap HP column (5 mL, GE Healthcare, Cat #28-9075) equilibrated with Strep binding buffer using an Äkta purification system (GE Healthcare). After washing, proteins were eluted in Strep binding buffer containing 2.5 mM desthiobiotin. Protein-containing fractions were pooled, desalted into Tris buffer (50 mM Tris, 250 mM NaCl, pH=8.0) using a 5 ml HiTrap Desalting column (GE Healthcare). Protein fractions were pooled and concentrated using MWCO centrifugal filters. Protein concentrations were measured on a Tecan infinite M200Pro plate reader by absorption at 280 nm (Nanoquant plate). Intact Protein MS was performed to confirm correct protein mass.

1.3 Gel-Based Labeling of PLPBP with PIxP Probes

Gel-based labelling of recombinant PLPBP was performed as described previously (Hoegl et al., 2018).

In brief, PLPBP (1 μM in PBS, total volume: 50 μL) was labeled with PLxP probe or mixture of PLxP probes (PL1P, PL2P, PL13P, final conc. 100 μM) at room temperature for 30 min. Upon reduction with 10 mM NaBH₄ (1 μl of 250 mM prepared fresh in 0.1 M NaOH) at room temperature for 30 min, proteins were precipitated by adding ice-cold acetone (4× volume) and incubating at −20° C. for at least 1 h. Precipitated proteins were pelletized by centrifugation (18,000×g, 15 min, 4° C.) and washed two times with ice-cold MeOH. For washing, protein pellet is resuspended in 0.2 mL cold methanol (−80° C.) by mild sonication (10 s, 10% intensity) and centrifuged (10 000 rpm, 10 min, 4° C.). Pelletized proteins were resuspended in 50 μl PBS containing 0.4% (w/v) SDS and Click chemistry was performed by adding 100 μM Cy5 azide (1 μL of 5 mM in DMSO), 100 μM TBTA (3 μL of 1.67 mM in/BuOH/DMSO 80:20), 1 mM TCEP (0.5 μL of 15 mg/mL stock in ddH₂O) and 1 mM CuSO₄ (1 μL of 50 mM stock in ddH₂O) to each sample and incubated for at least 1 h at r.t. Samples were quenched with 2× Lämmli buffer and analysed by SDS-PAGE (12.5% polyacrylamide gels) with subsequent fluorescence scanning.

1.4 Protein Overexpression of PLPBP and apoSHMT1

PLPBP and SHMT1 were recombinantly expressed as described previously (Fux et al., 2019; Fux and Sieber, 2019).

In brief, E. coli Rosetta2 (DE3) (Merck, Cat #71400) carrying the expression plasmids were cultured as described and expression was induced through the addition of 0.2 μg/mL anhydrotetracycline (ATET) for 2 h 37° C. Bacteria were harvested and washed with PBS (6,000×g, 4° C.). The cell pellet was resuspended in Strep binding buffer (50 mM NaH₂PO₄, 300 mM NaCl, pH=8.0) and lysed by sonication (see SaPLK purification). The lysate was clarified by centrifugation (36,000×g, 30 min, 4° C.). Supernatant was loaded onto a StrepTrap HP column (5 mL, GE Healthcare, Cat #28-9075) equilibrated with Strep binding buffer using an Äkta purification system (GE Healthcare). After washing, proteins were eluted in Strep binding buffer containing 2.5 mM desthiobiotin. For preparation of apo-SHMT1, the column was washed with 20 mL Strep binding buffer containing 25 mM hydroxylamine prior to elution.

Size exclusion chromatography (SEC) with Sepharose Superdex 75prep column (GE Healthcare) was applied for further purification. Proteins were loaded onto columns equilibrated with SEC buffer 20 mM HEPES, 100 mM KCl, pH=7.6). Protein fractions were pooled and concentrated using MWCO centrifugal filters. Protein concentrations were measured on a Tecan InfiniteM200 PRO plate reader (TECAN, Cat #IN-MNANO) by absorption at 280 nm (Nanoquant plate). Proteins were stored at −80° C. in small aliquots. Intact Protein MS was performed to confirm correct protein mass and PLP loading state of the enzymes.

### 1.5 Intact Protein MS (FIG. 11A)

Sample preparation: PLP loading state determination procedure was adapted from previous works by Pfanzelt et al. (2022). 40 μM apoSHMT1 (25 μl) were incubated with different equivalents of PLP (0.25 eq. 32.0 eq.) in PBS for 1 h at r.t. and subsequently treated with 20 mM NaBH₄ (2 μl of 250 mM stock prepared fresh in 0.1 M NaOH) for 30 min at r.t. For apoSHMT1 sample was directly reduced without prior incubation with PLP. NaBH₄ was quenched by acidification to pH=5-6 with 0.5% FA and neutralized to pH=7 with 0.1 M NaOH. Samples were diluted to 50 μl with PBS (20 μM final enzyme concentration) and transferred to MS vials for measurement via intact-protein MS.

IP-MS measurements were performed on an Ultimate 3000 RSLC system (Thermo Scientific) coupled to a LTQ Orbitrap XL mass spectrometer (Thermo Scientific). Protein desalting was carried out using a MassPREP desalting column (Waters) at 25° C. Gradient elution was carried out with 0.1% formic acid (LC-MS grade, Fisher Analytics) in water (LC-MS grade, Fisher Analytics) (A) and 0.1% formic acid in acetonitrile (MeCN, LC-MS grade, Fisher Analytics) (B). After 2 min pre-equilibration with 6% B, protein samples were injected and eluted with a linear gradient from 6% to 95% B over 1.5 min and 2 min at 95% B at 300 L/min flow rate. The column was re-equilibrated with 6% B for 1 min. Mass spectrometric measurements were conducted in HESI positive mode (H-ESI-II source, Thermo Scientific) with the following parameters: 4.0 kV capillary voltage, 350° C. capillary temperature, 31 V capillary voltage, 110 V tube lens, 30 L/h sheath gas, 15 L/h aux gas. Full scan measurements were accomplished in a range from 300-2000 m/z in profile mode in the orbitrap at a resolution of 100,000. Raw spectra were processed with UniDec 2.6.7 for deconvolution.

### 1.6 Preparation of Human Erythrocyte Lysate

Human erythrocytes were obtained from freshly drawn whole blood in EDTA tubes (4 ml each) via centrifugation (1×3500 rpm, 6 min, 1×3500 rpm, 8 min, 1×3000 rpm, 8 min). Supernatant (blood plasma) and buffy coat was removed and washed with PBS (5 mL) after each centrifugation step. To 3-4 mL erythrocytes were added 3-4 mL lysis buffer (Tris 50 mM, NaCl 150 mM, 1% NP40 (v/v), pH 7.5), incubated on ice for 5 min and frozen at −80° C. o/n. Thawed erythrocyte lysate was centrifuged (13 000 rpm, 10 min, 4° C.) and total protein amount is determined by BCA Assay (ROTI® Quant, Roth). Erythrocyte lysate is stored in small aliquots at −80° C. Blood samples were protected from light during preparation.

### 1.7 Sample Preparation for Microarray Analysis

### Workflow 1 (Initial Workflow) (FIG. 7)

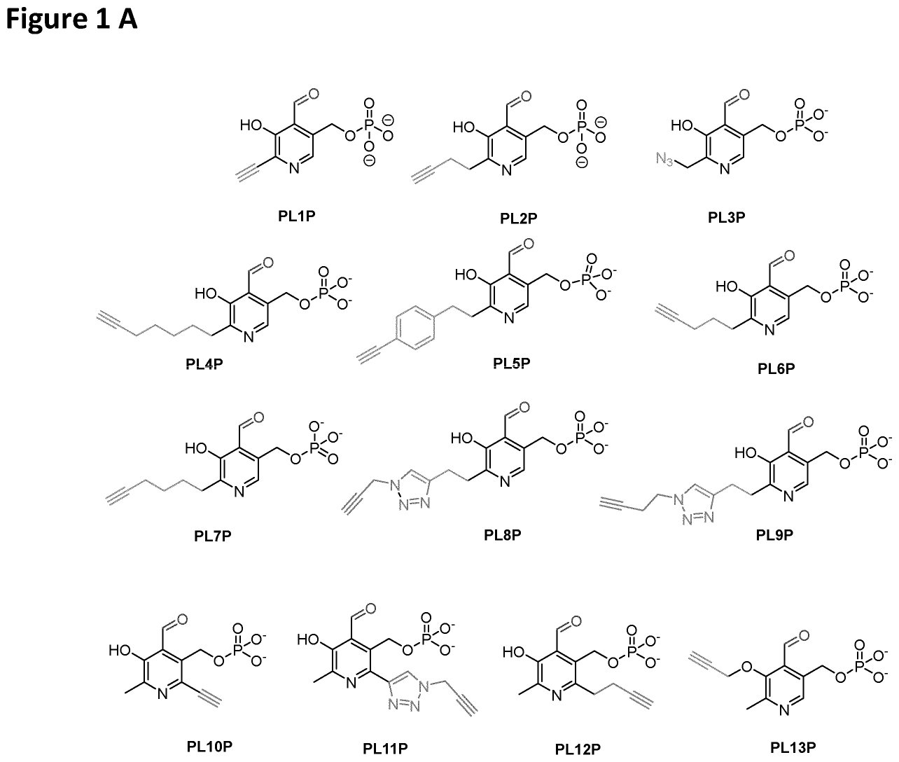

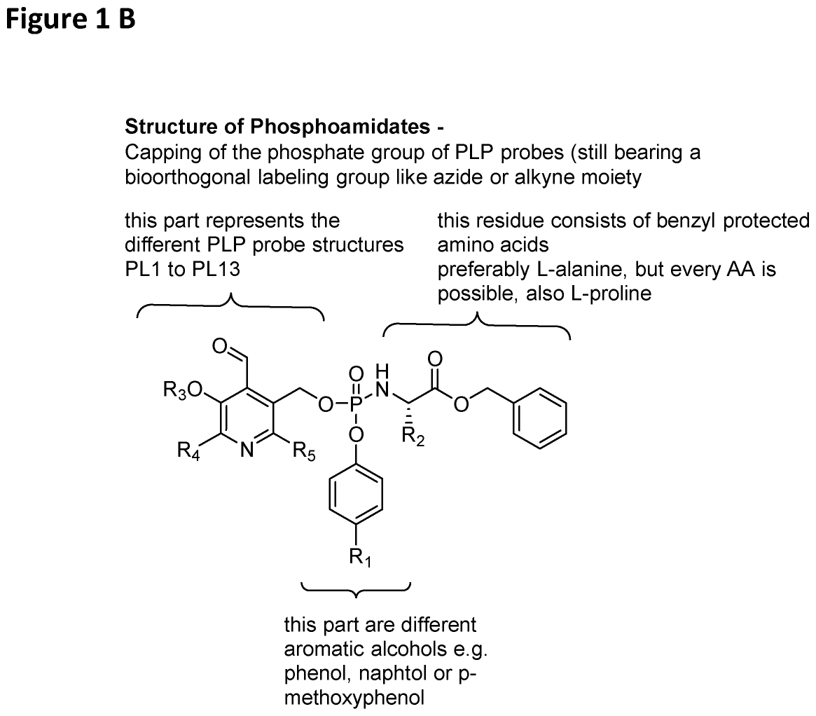

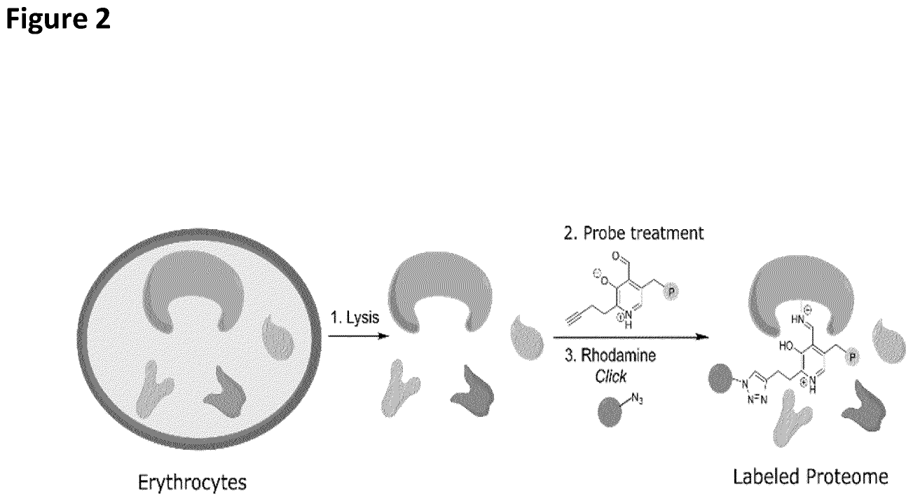

In 100 μL PBS, recombinant PLPBP (final conc. 6 μM) was incubated with 100 μM PL2P probe for 1 h at r.t. Labeled recombinant protein was treated with 20 mM NaBH₄ (250 mM stock solution in 0.1 M NaOH) and incubated for 30 min at r.t. Reduction was quenched by adding cold acetone (4×volume of sample, −20° C.) for at least 1 h. Precipitated protein was pelletized (10 000 rpm, 15 min, 4° C.) and supernatant was discarded. For washing, protein pellet was resuspended in 0.5 mL cold methanol (−80° C.) by mild sonication (10 s, 10% intensity) and centrifuged (10 000 rpm, 10 min, 4° C.). Each protein pellet was resuspended in 100 μL PBS by sonication (10 s, 10% intensity). The samples were subjected to Click reaction via addition of 100 μM Cy5 azide (Figure S1) or 100 μM Rh—N₃ (FIG. 2A) (1 μL of 10 mM stock in DMSO), 100 μM TBTA (1.667 mM stock in/BuOH/DMSO 80:20), 1 mM TCEP (15 mg/mL stock in ddH₂O) and 1 mM CuSO₄ (50 mM stock in ddH₂O) to each sample (100 μL) and incubated for 1 h at r.t. Click reaction was quenched by adding cold acetone (4× volume of sample, −20° C.) and stored for 1 h at −20° C. Precipitated protein was pelletized (10 000 rpm, 15 min, 4° C.) and supernatant was discarded. For washing, protein pellet was resuspended in 0.5 mL cold methanol (−80° C.) by mild sonication (10 s, 10% intensity) and centrifuged (10 000 rpm, 10 min, 4° C.). Finally, each protein pellet was resuspended in 100 μL 2% Tween20. Samples were subsequently incubated and analysed on the microarrays.

### Workflow 2 (Improved Workflow) (FIGS. 8, 9, 10, 12)

For erythrocyte lysate samples that were not depleted with Ni-NTA beads, 0.5 μL of erythrocyte lysate were diluted in 100 μL PBS. Recombinant PLP-DEs were diluted in PBS according to the desired concentration. For hemoglobin-depleted erythrocyte lysate samples, following general procedure was performed prior to labelling with PL2P:

General Procedure for Hemoglobin Depletion of Erythrocyte Lysate (EL) with Ni-NTA Beads

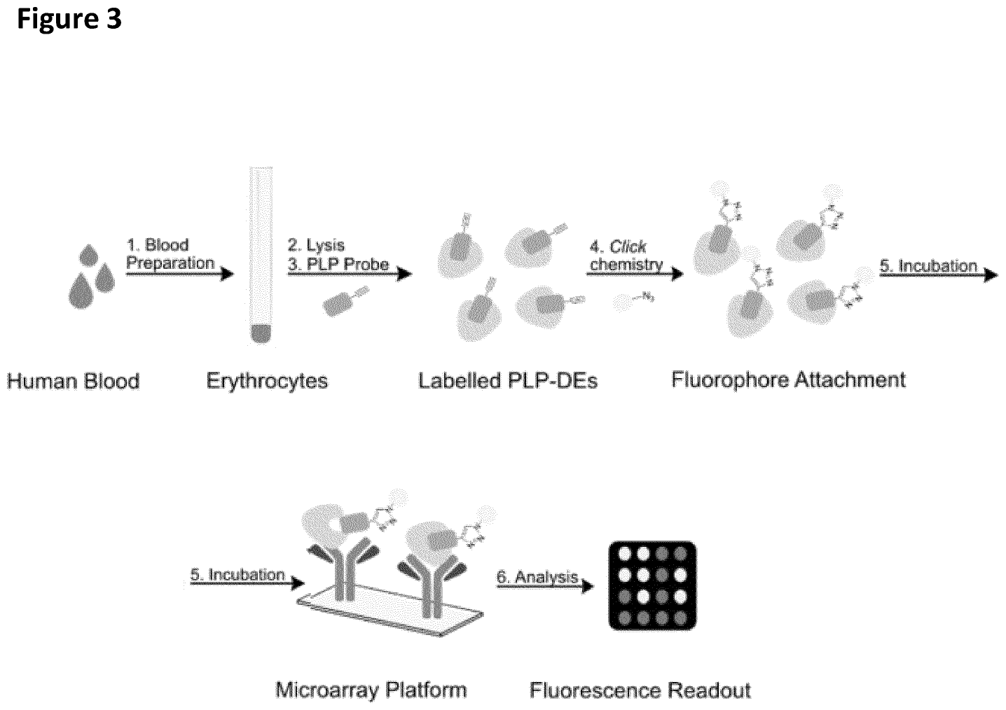

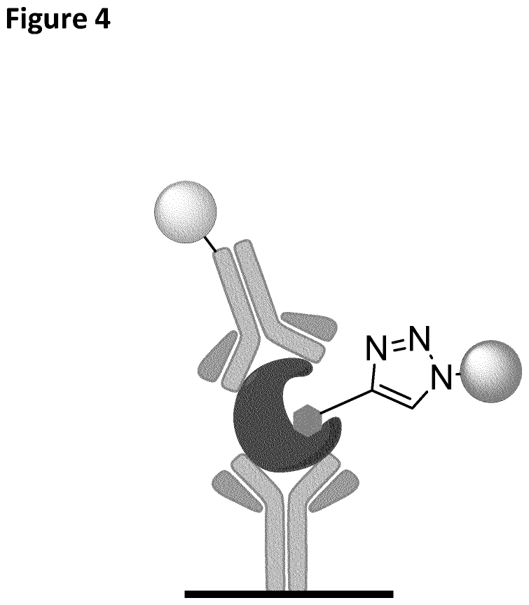

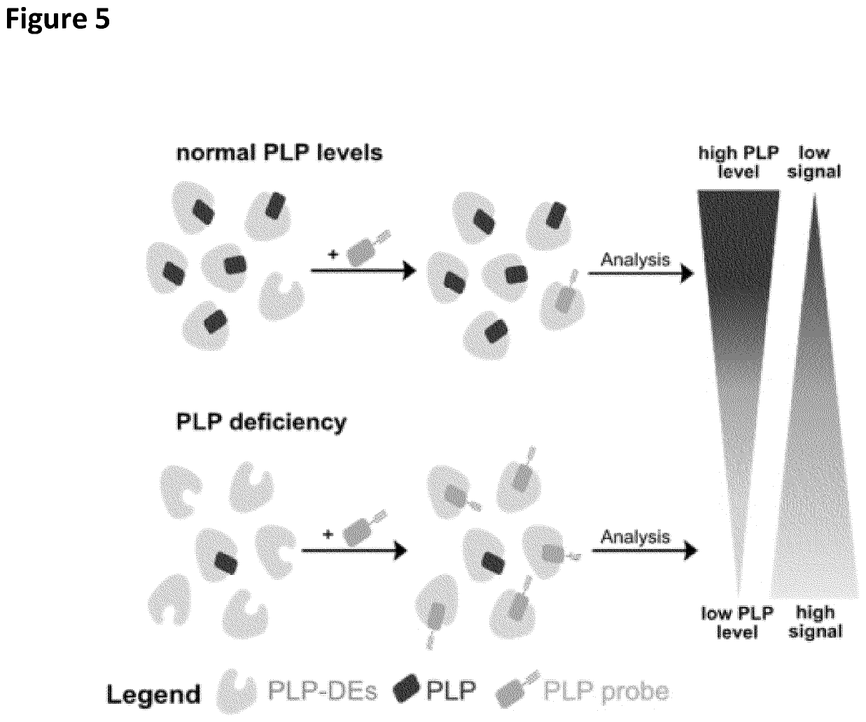

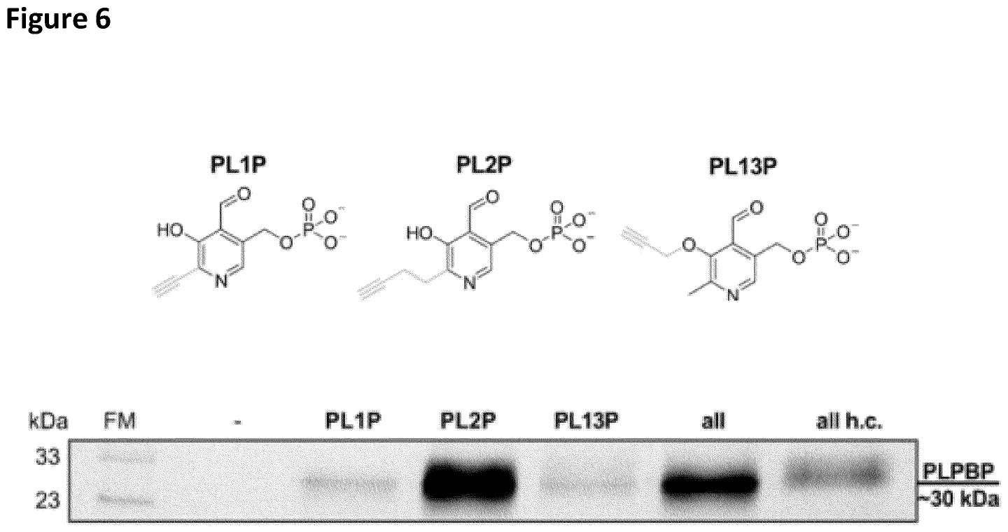

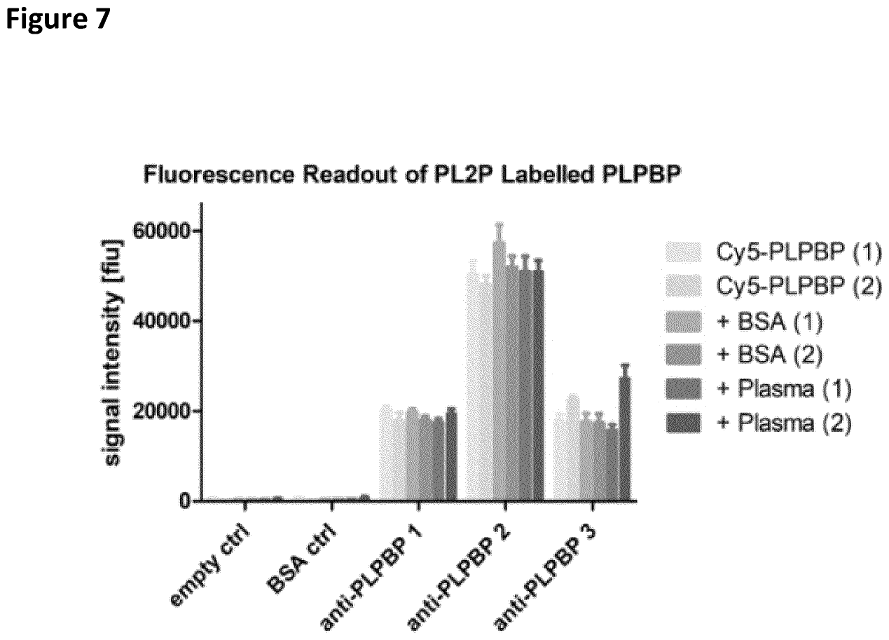

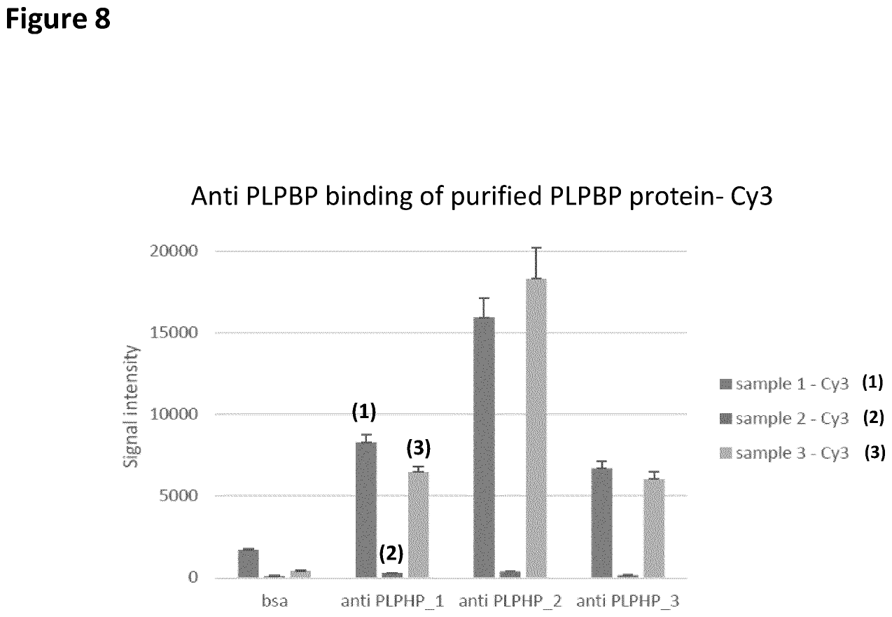

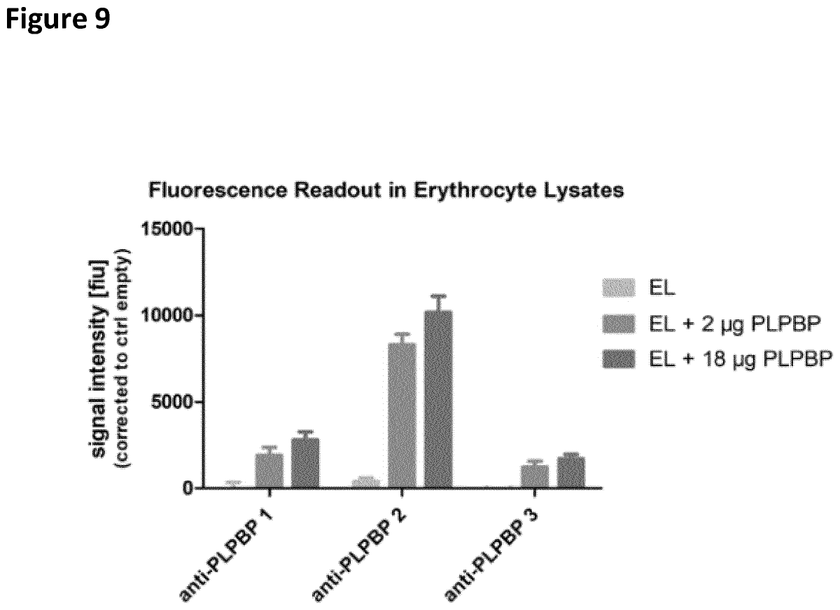

Ni-NTA agarose beads (Qiagen) were carefully resuspended by inversion on an Eppendorf tube wheel at 4° C. and transferred to an Eppendorf tube (X×100 μL+25% in one LoBind Eppendorf tube with marked filling level before washing steps are performed). The beads were washed with PBS (3×1 mL), centrifuged (400 g, 3 min, r.t.) and filled to the original level with PBS. For each sample, 100 μL of bead suspension (50% PBS, 50% beads) were incubated with 7 μL erythrocyte lysate for 30 min at r.t. The samples were transferred quantitatively onto spin columns (Biokcho, non-filled) and centrifuged (1000× g, 2 min, r.t.). Collected flow through was filled up to a final volume of 100 μL with PBS and further subjected to probe labelling. In FIG. 3A, 14 μL erythrocyte lysate and 100 μL Ni-NTA beads were used for sample preparation of 2× EL.

### Sample Preparation

Different amounts of recombinant PLP-DE or erythrocyte lysate in 100 μL PBS were incubated with 40 μM or 100 μM (Figure S1, 2A) PL2P probe for 1 h at r.t. The samples are subjected to Click reaction via addition of 100 μM rhodamine azide (1 μL of 10 mM stock in DMSO), 100 μM TBTA (6 μL of 1.667 mM stock in/BuOH/DMSO 80:20), 1 mM TCEP (1 μL of 15 mg/mL stock in ddH₂O) and 1 mM CuSO₄ (1 μL of 50 mM stock in ddH₂O) to each sample (100 μL) and incubated for at least 1 h at r.t. Samples are subsequently incubated and analysed on the microarrays.

Workflow 3 (Workflow for Experiment with apoSHMT1, FIG. 11B)

40 μM apoSHMT1 (25 μL) samples were incubated with increasing amounts of PLP (0 eq., 0.50 eq., 1.00 eq., 4.00 eq., 32.0 eq.) for 1 h at r.t. and subsequently treated with 20 mM NaBH₄ (250 mM stock solution in 0.1 M NaOH) for 30 min at r.t. Reduction was quenched by adding cold acetone (4× volume of sample, −20° C.) for at least 1 h. Precipitated proteins were pelletized (10 000 rpm, 15 min, 4° C.) and supernatant was discarded. For washing, protein pellet was resuspended in 0.1 mL cold methanol (−80° C.) by mild sonication (10 s, 10% intensity) and centrifuged (10 000 rpm, 10 min, 4° C.). Each protein pellet was resuspended in 100 μL PBS by sonication (10 s, 10% intensity) and incubated with 100 μM PL2P probe for 1 h at r.t. The samples are subjected to Click reaction via addition of 100 μM rhodamine azide (1 μL of 10 mM stock in DMSO), 100 μM TBTA (6 μL of 1.667 mM stock in/BuOH/DMSO 80:20), 1 mM TCEP (1 μL of 15 mg/mL stock in ddH₂O) and 1 mM CuSO₄ (1 μL of 50 mM stock in ddH₂O) to each sample (100 μL) and incubated for at least 1 h at r.t. Samples are subsequently incubated and analysed on the microarrays. Value with 32.0 eq. PLP got excluded in further analysis because of too high background signals in empty and BSA controls.

### 1.8 Microarray Production

Custom microarrays were produced using the automated production facility at Sciomics. Standard production protocols using contact printing on chemically modified glass surfaces (Epoxy-coupling groups) were used and slides were individually quality controlled after production. Produced Slides were stored according to Sciomics SOPs cold and protected from light.

### 1.9 Antibody Labelling

The secondary antibodies were labelled at an adjusted protein concentration for two hours with scioDye2 (Sciomics, Germany). After two hours the reaction was stopped and the buffer exchanged to PBS. All labelled protein samples were stored at −20° C. until use.

### 1.10 Samples and Protein Extraction

The samples were used immediately after performing Click chemistry without any freeze/thaw cycles and only minimal storage times at 4° C.

### 1.11 Sample Incubation

The samples were analysed in a single colour approach (without secondary antibody incubation) or dual-colour approach (with secondary antibody incubation) using custom microarrays containing antibodies against OAT, SHMT and PLPBP or a subset of these antibodies. Each antibody is represented on the array in 8 replicates (array format with PLPBP, SHMT1 and OAT antibodies) or 16 replicates (array format with three PLPBP antibodies). The arrays were blocked with scioBlock (Sciomics) on a Hybstation 4800 (Tecan, Austria) and afterwards the samples were incubated. After incubation for three hours, the slides were either thoroughly washed with 1× PBSTT, rinsed with 0.1× PBS as well as with water and subsequently dried with nitrogen or incubated with the fluorescently labelled secondary antibody for one hour, followed by washing and drying as described above.

### 1.12 Data Acquisition and Analysis

Slide scanning was conducted using a Powerscanner (Tecan, Austria) with constant instrument laser power and PMT settings. Spot segmentation was performed with GenePix Pro 6.0 (Molecular Devices, Union City, CA, USA). Acquired raw data were analysed using Microsoft Excel for all calculations such as average signal values and standard deviations indicated as error bars in figures. Data visualization was performed using GraphPad Prism 5. Signals were baseline corrected (signal-baseline) to mean values of either empty control (ctrl empty) or BSA control (ctrl BSA). Signal ratios in FIG. 12B were obtained by dividing mean values of PL2P signal by mean values of PL2P second labelled antibody signal (PL2P/total PLPBP).

### 1.13 Determination of Vitamin B6 Value in Human Blood (Whole Blood or Erythrocytes) Using Standard HPLC Diagnostics

Sample preparation for the routine determination of B6 in its active coenzyme form from EDTA whole blood was performed according to the manufacture's protocol (Vitamin B6 in Whole Blood/Plasma-HPLC Kit, Chromsystems). In brief, in a light protected vial 200 μl whole blood was mixed with 100 μl Internal Standard and 300 μl Precipitation Reagent for 30 seconds and centrifuged for 5 min at 14000× g. In a new light protected vial, 250 μl Neutralisation Reagent and 100 μl Derivatisation Mix are mixed shortly with 250 μl of supernatant obtained above and incubated for 25 min at 60° C. resulting in a fluorescent pyridoxal-5′-phosphate (PLP) compound. The HPLC measurement was performed isocratically with fluorescence detection (excitation wavelength 320 nm, emission wavelength 415 nm) using a whole blood calibration standard (Chromsystems).

### Example 2 Results

### 2.1 Design of the PLP-Detection Platform

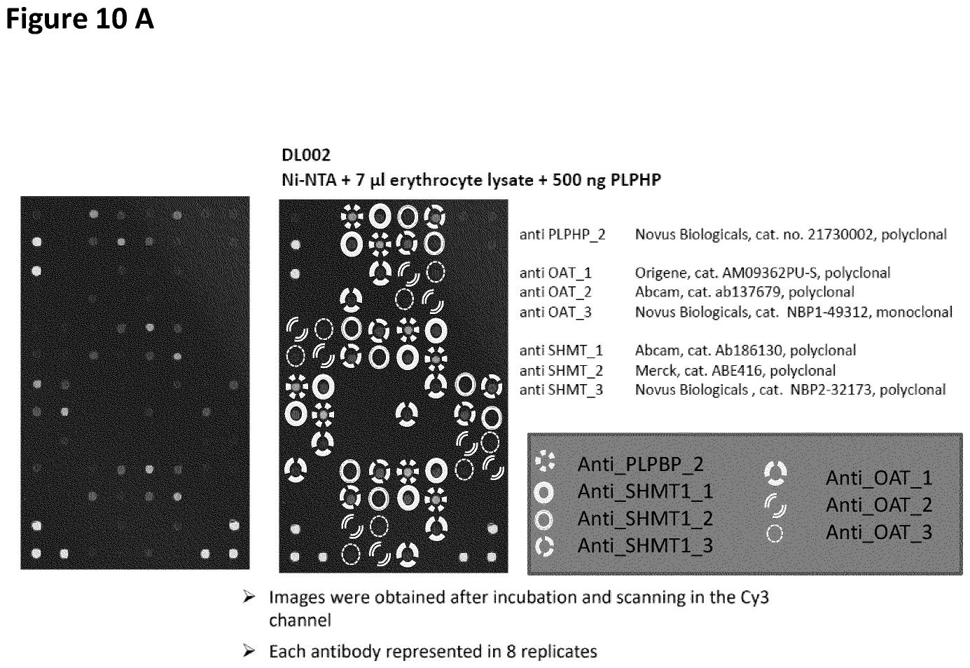

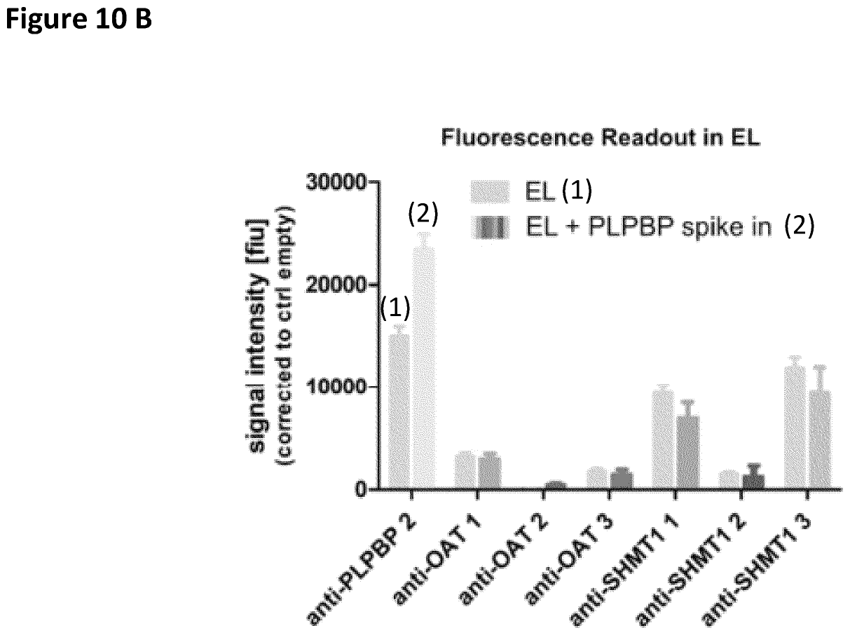

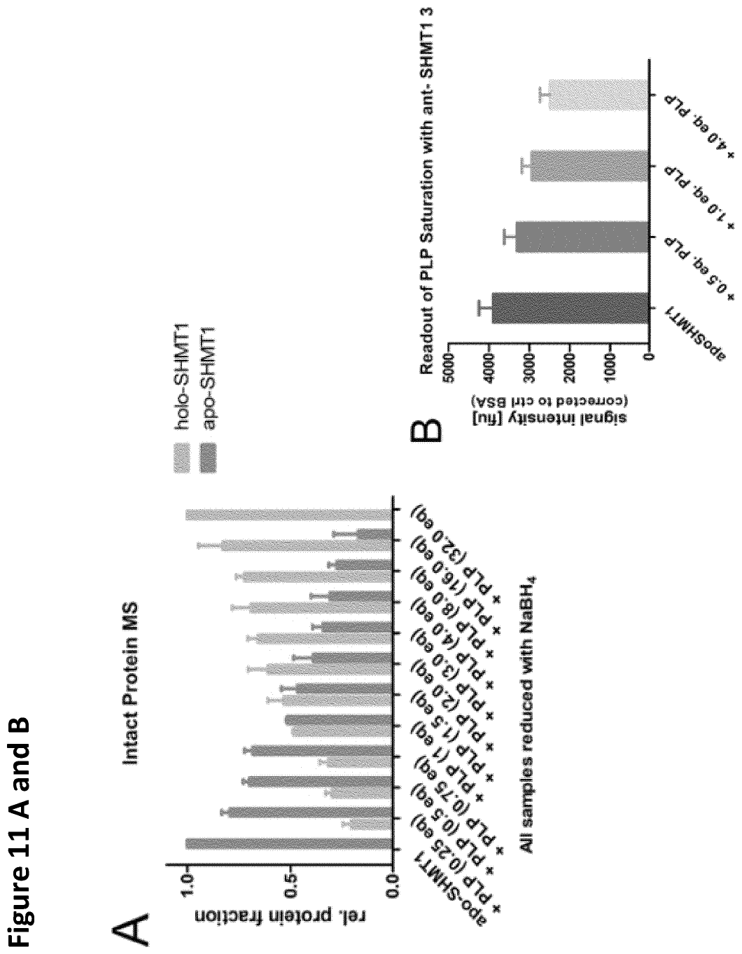

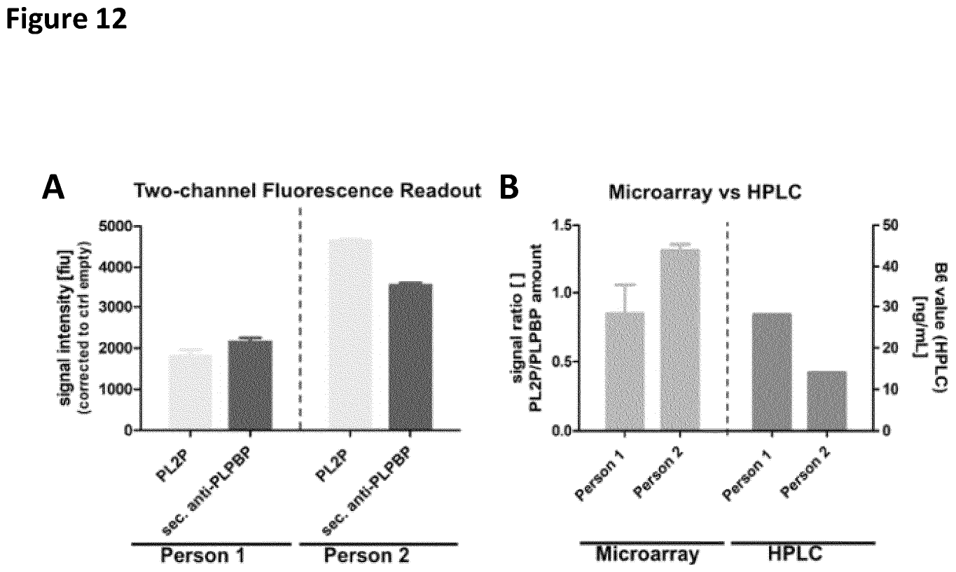

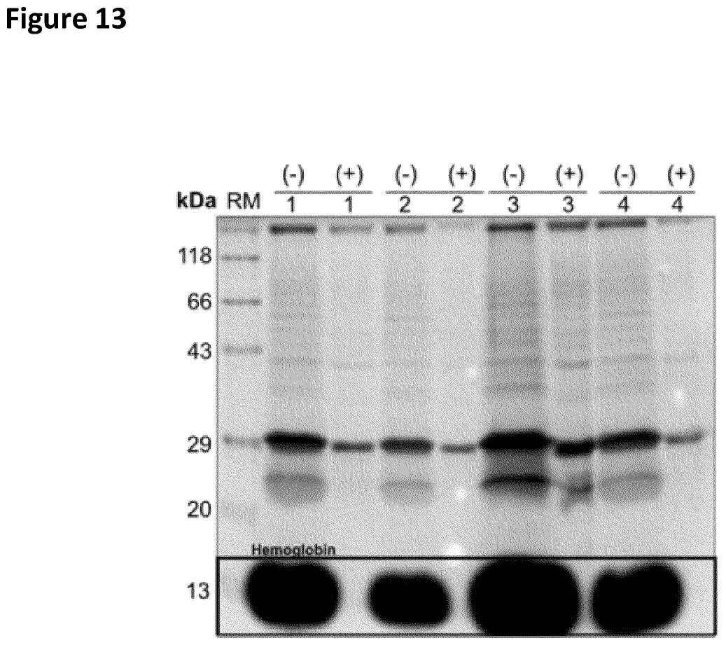

The design of the B6-detection platform followed the need for an easy, reliable and rapid readout. We thus devised a strategy by which erythrocytes, isolated from human blood, are lysed followed by treatment with a PLP probe to occupy free PLP-DE cofactor binding sites and Click chemistry to rhodamine azide (Meldal et al., 2008). Next, labeled proteins are captured by specific antibodies on custom produced microarrays and the corresponding signals are analyzed via fluorescence readout (see FIG. 3). A high signal indicates a low saturation with endogenous PLP (many unoccupied sites and high probe binding) while a low signal corresponds to sufficient PLP supplementation (many occupied sites and low probe binding, FIG. 4). First, we identified suitable signature PLP-DEs, present in erythrocytes, as reliable measures for the B6 status. Of the enzymes expressed, we selected pyridoxal 5′-phoshate binding protein (PLPBP, formerly known as PROSC), serine hydroxymethyl transferase 1 (SHMT1) and ornithine amino transferase (OAT1) based on their relevance in PLP homeostasis, metabolism and disease (Renwick et al., 1998, Darin et al., 2016, Roessler et al., 2012, Fux & Sieber, 2019, Ginguay et al., 2017) Furthermore, to cover a broad dynamic range all three PLP-DEs are of either high, medium or low abundance in erythrocytes as determined in previous proteomic studies (Bryk et al., 2017).

a) Recombinant Labelling of Human PLPBP with Different Functionalized PLP Cofactors.

Next, we tested our library of pyridoxal probes/PLP cofactor mimics for the most comprehensive labeling.

For this, we selected recombinant human PLP binding protein (PLPBP, UniProt 094903) as a medium abundant model PLP-DE of human erythrocytes.

Conditions: 1 μM PLPBP, 100 μM probe (incubation 1 h, r.t.), reduction (NaBH₄), precipitation (aceton), wash (MeOH), redissolve in PBS+0.2% SDS, click to Cy5. Analysis via fluorescence SDS-Page. Results see FIG. 6.

The following three PLP cofactor mimics were successfully tested: PL1P, PL2P and PL13P, wherein PL2P was identified as the most suitable PLP cofactor mimic for labeling of recombinant PLPBP via SDS-Page (see FIG. 6).

### b) Anti-PLPBP Antibodies

Next, we produced a prototype microarray with three candidate PLPBP antibodies to select the best capture antibody and to establish the general workflow. The following three different anti-PLPBP antibodies tested:
anti PLPHP 1: Thermofischer, cat. nr. PA532036, polyclonal
anti PLPHP_2: Novusbio, cat. nr. 21730002, polyclonal
anti PLPHP_3: Origene, cat. nr. TA505130, monoclonal

Antibodies were printed on chemically modified glass surfaces (modified epoxy-coupling groups to enable a covalent attachment of the antibodies) using contact printing technique (Sciomics GmbH, Germany). The antibodies were immobilized on glass sides, which contain several replicates of the antibody for quality control and statistical evaluation. Recombinant PLPBP incubated with PL2P and clicked to Cy5 was used. Results see FIG. 7 showing that all three immobilized antibodies against PLPBP work.

Recombinant PLPBP was labeled with PL2P, the reversible internal aldimine bond reduced with NaBH₄, clicked to the Cy5-azide fluorescent dye, precipitated, washed, and redissolved in buffer prior to incubation on the microarray platform. As can be seen in FIG. 7, PL2P-labeled PLPBP was detected by all three antibodies, also in presence of plasma or BSA as matrix background. Of note, one antibody exhibited a 50% superior intensity of detection which was selected for further studies (FIG. 7).

c) In General, Overall Concept and Microarray Works with Good S/N Ratios. Plasma and BSA do not Interfere with the Workflow and Fluorescent Analysis.

The experimental conditions were further optimized to a sample preparation, where no reduction of the internal aldimine to the secondary amine, no precipitation and removal of excess dye is necessary prior to the incubation on the microarray with subsequent fluorescent analysis. Additionally, negative (sample 2) and positive (sample 3) controls verify the overall concept for recombinant PLPBP detection on our microarray with immobilized antibodies against PLPBP. Results see FIGS. 7 and 8.

We were able to detect recombinant PLPBP down to a concentration of 31.3 ng (data not shown) which is suitable for monitoring endogenous enzyme concentrations. These pilot experiments demonstrate the robustness, ease of use, sensitivity, speed and reliability of the detection platform-ready to use in erythrocyte samples.

### d) Signal Intensity Dependent on PLP Saturation of Enzyme.

To further validate the method of invention, it was proven that the obtained fluorescent signal is dependent on the PLP saturation state of the enzyme. Hereby, serine hydroxymethyltransferase 1 was recombinantly expressed as the apo-enzyme and gradually saturated with different amounts of PLP, that was irreversibly bound to the SHMT1 enzyme via NaBH₄ to ensure a fixed saturation state (verified by IP-MS). The observed decrease in signal intensity with increasing PLP saturation shown in FIG. 11 is in accordance with our inverse correlation theory that less PL4P probe can bind to a higher PLP saturated enzyme.

### 2.2 Microarrays Reliably Monitor PLP-DEs in Erythrocytes

We commenced our studies by detecting probe labeling of endogenous PLPBP in human erythrocyte lysates (ELs). While the spike-in of recombinant PLPBP in the proteome followed by probe labeling yielded clear signals on the array, we were unable to detect endogenous PLPBP (FIG. 9). A possible reason for this low sensitivity are high amounts of hemoglobin and carboanhydrase-1 which both account for up to 98% of the total soluble protein amount in erythrocytes (Ringrose et al., 2008) leading to high background fluorescence. We thus adapted literature procedures by Ringrose et al. (2008) and Yang et al. (2012) to deplete hemoglobin in erythrocyte lysate via Ni-NTA beads prior to incubation with the PL2P probe (FIG. 13, SDS Page hemoglobin depletion).

Satisfyingly, this additional background reduction step enabled the successful detection of endogenous PLPBP in erythrocytes. Increasing the amount of EL used and spike-in of recombinant PLPBP to the erythrocyte lysate additionally confirmed a concentration dependent readout of the corresponding signal intensity which validates the platform for the reliable B6 status assessment in blood samples. Results see FIG. 9. This is also visible in the obtained microarray images (see FIG. 10A), where human erythrocyte lysate was also spiked with PLPBP. This image additionally shows the extension of our microarray format to several PLP-DEs. In total, 7 different antibodies targeting the three PLP-DEs PLPBP, SHMT1 and OAT showed very good to moderate signal intensities proving that the array can be exploited for all PLP-DEs if suitable antibodies are available. In total, probe labeling of the three signature enzymes in erythrocytes showed reliable signal-to-noise ratios in all cases (FIG. 10B).

### 2.3 Assessment of the B6 Status in Human Blood Samples

One of the major tasks of this array format is the direct assessment of B6 supplementation in human blood. A major prerequisite for this goal is the correct readout of accessible binding sites in PLP-DEs which will differ based on the B6 availability. To approach this task, recombinant apoSHMT1 was pre-incubated with increasing amounts of PLP followed by analysis of the saturation status first via intact mass-spectrometry (IP-MS) and second via the array platform. In line with the increase of PLP binding to SHMT1, as confirmed by MS (FIG. 11A), a decline in signal intensity of the array was observed, demonstrating less accessible sites for probe binding (FIG. 11B).

A second prerequisite to ensure a reliable readout is the normalization of the fluorescent signal to the total protein amount. For example, if a PLP-DE is of higher abundance in an individual it will give a more intense signal than an individual with lower abundance of this enzyme at the exact same level of B6 saturation. To account for these individual-specific differences we devised a sandwich-based assay format in which a second fluorescent antibody (bearing a different fluorophore) with specificity for the signature enzyme is added after incubation of the microarray with probe treated lysate (FIG. 4). Herein, a two-channel fluorescence readout would reveal high ratios of probe/protein in case of a low vitamin B6 status and vice versa. We obtained blood samples of two healthy volunteers which we first analyzed with the classical B6 assessment via laboratory HPLC assays (from EDTA whole blood). The tested individuals varied in their PLP concentration from 14 ng/mL to 28 ng/ml which is both within the reference range of healthy PLP levels, given by a routine HPLC method (˜13-28 ng/ml, 55-110 nmol/L)³⁸. The respective fluorescence signals and ratios of both volunteers according to the Sandwich fluorescent readout are shown in FIG. 12A and B. A lower ratio of the two fluorescence signals in the sample from Person 1 demonstrates less PL2P probe binding and thus refers to a higher saturation of the PLP-DE with vitamin B6 compared to Person 2. Vice versa, the high ratio of signals in the sample from Person 2 points to a lower vitamin B6 saturation level compared to Person 1. Importantly, these results are in accordance with the predetermined vitamin B6 HPLC values of the volunteers (FIG. 12B).

The features disclosed in the foregoing description, in the claims and/or in the accompanying drawings may, both separately and in any combination thereof, be material for realizing the invention in diverse forms thereof.

The project leading to this application has received funding from the European Research Council (ERC) under the European Union's Horizon 2020 research and innovation program (grant agreement No. 875594).

### REFERENCES

Brussaard, J., Löwik, M., Van den Berg, H., Brants, H. & Bemelmans, W. Dietary and other determinants of vitamin B6 parameters. European journal of clinical nutrition 51, S39-45 (1997).
Bryk, A. H. & Wiśniewski, J. R. Quantitative analysis of human red blood cell proteome. Journal of proteome research 16, 2752-2761 (2017).
CDC. Second National Report on Biochemical Indicators of Diet and Nutrition in the U.S. Population. 495 (Center for Disease Control and Prevention, 2012).
Dalery, K. et al. Homocysteine and coronary artery disease in French Canadian subjects: relation with vitamins B12, B6, pyridoxal phosphate, and folate. The American journal of cardiology 75, 1107-1111 (1995).
Darin, N. et al. Mutations in PROSC Disrupt Cellular Pyridoxal Phosphate Homeostasis and Cause Vitamin-B (6)-Dependent Epilepsy. Am J Hum Genet 99, 1325-1337 (2016).
Driskell, J. A. in Nutrition Research Vol. 14, 293-324 (1994)
Fux, A., Pfanzelt, M., Kirsch, V. C., Hoegl, A. & Sieber, S. A. Customizing Functionalized Cofactor Mimics to Study the Human Pyridoxal 5′-Phosphate-Binding Proteome. Cell Chemical Biology 26, 1461-1468. e1467 (2019).
Fux, A. & Sieber, S. A. Biochemical and Proteomic Studies of Human Pyridoxal 5′-Phosphate-Binding Protein (PLPBP). ACS Chemical Biology 15, 254-261 (2019).
Ginguay, A., Cynober, L., Curis, E. & Nicolis, I. Ornithine aminotransferase, an important glutamate-metabolizing enzyme at the crossroads of multiple metabolic pathways. Biology 6, 18 (2017).
Gregory, J. & Kirk, J. Vitamin B6 in foods: assessment of stability and bioavailability. (1977).
Hoegl, A. et al. Mining the cellular inventory of pyridoxal phosphate-dependent enzymes with functionalized cofactor mimics. Nature chemistry 10, 1234-1245 (2018).
Huang, Y. C., Chen, W., Evans, M. A., Mitchell, M. E. & Shultz, T. D. Vitamin B-6 requirement and status assessment of young women fed a high-protein diet with various levels of vitamin B-6. The American journal of clinical nutrition 67, 208-220 (1998).
Hustad, S. et al. Kinetic modeling of storage effects on biomarkers related to B vitamin status and one-carbon metabolism. Clinical chemistry 58, 402-410 (2012).
Johansson, M. et al. Serum B vitamin levels and risk of lung cancer. Jama 303, 2377-2385 (2010).
Kanchi, S., Sabela, M. I., Mdluli, P. S., Inamuddin & Bisetty, K. Smartphone based bioanalytical and diagnosis applications: A review. Biosensors and Bioelectronics 102, 136-149 (2018).
Kelly, P. et al. Inflammation, homocysteine, and vitamin B6 status after ischemic stroke. Stroke 35, 12-15 (2004).
Leklem, J. E. Vitamin B-6: a status report. The Journal of nutrition 120, 1503-1507 (1990).
Lotto, V., Choi, S. W. & Friso, S. Vitamin B6: a challenging link between nutrition and inflammation in CVD. British journal of nutrition 106, 183-195 (2011).
Meldal, M. & Tornøe, C. W. Cu-Catalyzed Azide-Alkyne Cycloaddition. Chemical Reviews 108, 2952-3015, doi: 10.1021/cr0783479 (2008).
Panton, K. K., Farup, P. G., Sagen, E., Sirum, U. F. & Åsberg, A. Vitamin B6 in plasma-sample stability and the reference limits. Scandinavian journal of clinical and laboratory investigation 73, 476-479 (2013).
Pfanzelt, M., Maher, T. E., Absmeier, R. M., Schwarz, M., Sieber, S. A. Tailored Pyridoxal Probes Unravel Novel Cofactor-Dependent Targets and Antibiotic Hits in Critical Bacterial Pathogens. Angewandte Chemie (2022) https://doi.org/10.1002/anie.202117724
Renwick, S. et al. Purification, crystallization and preliminary X-ray analysis of human recombinant cytosolic serine hydroxymethyltransferase. Acta Crystallographica Section D: Biological Crystallography 54, 1030-1031 (1998).
Ringrose, J. H. et al. Highly Efficient Depletion Strategy for the Two Most Abundant Erythrocyte Soluble Proteins Improves Proteome Coverage Dramatically. Journal of Proteome Research 7, 3060-3063, doi: 10.1021/pr8001029 (2008).
Reynolds, R. D. in Vitamin B-6 Metabolism in Pregnancy, Lactation, and Infancy 41-59 (CRC Press, 1995).
Rimm, E. B. et al. Folate and vitamin B6 from diet and supplements in relation to risk of coronary heart disease among women. Jama 279, 359-364 (1998).
Robinson, K. et al. Low circulating folate and vitamin B6 concentrations: risk factors for stroke, peripheral vascular disease, and coronary artery disease. Circulation 97, 437-443 (1998).
Roessler, S. et al. Integrative genomic identification of genes on 8p associated with hepatocellular carcinoma progression and patient survival. Gastroenterology 142, 957-966. e912 (2012).
Sies, H., Stahl, W. & Sundquist, A. R. Antioxidant functions of vitamins: Vitamins E and C, Beta-Carotene, and other carotenoids a. Annals of the New York Academy of Sciences 669, 7-20 (1992).
Ueland, P. M., Ulvik, A., Rios-Avila, L., Midttun, Ø. & Gregory, J. F. Direct and functional biomarkers of vitamin B6 status. Annual review of nutrition 35, 33-70 (2015).
Van der Ham, M. et al. Quantification of vitamin B6 vitamers in human cerebrospinal fluid by ultra performance liquid chromatography-tandem mass spectrometry. Analytica chimica acta 712, 108-114 (2012).
Vanuzzo, D. et al. Both vitamin B6 and total homocysteine plasma levels predict long-term atherothrombotic events in healthy subjects. European heart journal 28, 484-491 (2007).
Verhoef, P. et al. Homocysteine metabolism and risk of myocardial infarction: relation with vitamins B6, B12, and folate. American journal of epidemiology 143, 845-859 (1996).
Vermaak, W. et al. Vitamin B-6 nutrition status and cigarette smoking. The American journal of clinical nutrition 51, 1058-1061 (1990).
Yang, H., Zhou, B., Prinz, M. & Siegel, D. Proteomic Analysis of Menstrual Blood*. Molecular & Cellular Proteomics 11, 1024-1035, (2012).

1. An in vitro method for determining vitamin status of a subject, wherein said method comprises:
(a) harvesting erythrocytes from a sample obtained from a subject or providing erythrocytes obtained from a sample of a subject;
(b) providing a probe comprising at least one functionalized cofactor of PLP-dependent enzymes (PLP-DEs) (PLP probe);
(c) lysing the erythrocytes and thereby releasing the proteome including the PLP-DEs and then treating the lysate with the PLP probe provided in (b); or
(c′) treating the erythrocytes with the PLP probe provided in (b) and then lysing the erythrocytes and thereby releasing the proteome including the PLP-DEs;
(d) adding a label for the PLP-probe to the treated lysate of (c) or to the lysate of (c′);
(e) capturing the labeled PLP-DEs with anti-PLP-DE-antibodies immobilized on a surface, preferably an array or a membrane,
(f) detecting the labeled PLP-DEs, and
(g) determining the vitamin status of the subject.

2. The method of claim 1, wherein in step (a) the erythrocytes are harvested by centrifugation and the sample is whole blood.

3. The method of claim 1, wherein the cofactor of PLP-DEs is functionalized with a biorthogonal labeling group,
wherein preferably the biorthogonal labeling group is selected from an alkyne or azide moiety or a tetrazine functionality,
wherein the cofactor of PLP-DEs is optionally further functionalized with a phosphoamidate.

4. The method of claim 3, wherein the biorthogonal labeling group comprises an alkyne group,
and the cofactor probe is selected from PL1P, PL2P, PL4P to PL13P,
or wherein the biorthogonal labeling group is an azide group, and the cofactor probe is preferably PL3P.

5. The method of claim 1, wherein the probe comprises at least two functionalized cofactors of PLP-DEs,
and/or wherein the probe comprises a further moiety or tag.

6. The method of claim 1, wherein the lysis of the erythrocytes in step (c) or in step (c′) comprises the removal of hemoglobin.

7. The method of claim 1, wherein the treatment in step (c) comprises an incubation of the lysate with the PLP probe,
or wherein the treatment in step (c′) comprises an incubation of the erythrocytes with the PLP probe.

8. The method of claim 1, wherein the label in step (d) is
a fluorescent label,
a bioluminescence label,
a chemiluminescence label,
a dye,
or a combination thereof
wherein the label is attached to the biorthogonal labeling group of the cofactors.

9. The method of claim 1, wherein in step (e) second anti-PLP-DE-antibodies are added, wherein said second anti-PLP-DE-antibodies are labelled,
wherein the label of said second anti-PLP-DE-antibodies is different to the label of step (d) and is selected from a fluorescent label, a bioluminescence label, a chemiluminescence label, a dye, or a combination thereof;
wherein in step (f) the label of said second anti-PLP-DE-antibodies is detected in addition to the signal of the labeled PLP-DEs and a ratio of PLP loading state/total PLP-DE amount is obtained by comparing the signal of the labeled PLP-DEs and the signal of the second antibodies.

10. The method of claim 1, wherein in step (g) the vitamin status is determined by comparing the readout of step (f) with a control,
and/or wherein the method is a point of care method.

11. The method of claim 1, further comprising:
dividing the sample obtained from the subject in two parts and pre-treating one part with a PLP standard prior to step (c),
performing steps (a) to (f) for both parts in parallel,
comparing the results of step (f) for both parts and thereby quantifying the PLP status.

12. The method of claim 1, wherein the vitamin status determined in a subject is used for:
determining the vitamin B6 status of the subject,
monitoring the PLP status and/or vitamin B6 status of the subject, and/or
diagnosing vitamin B6-deficiency and vitamin B6-deficiency-associated diseases.

13. A kit for in vitro determining vitamin status in a sample of a subject, comprising:
(1) a probe comprising at least one functionalized cofactor of PLP-dependent enzymes (PLP-DEs) (PLP probe);
(2) a label for the PLP-probe;
(3) optionally, an array for PLP-DEs; and
(4) optionally, reagents for lysing erythrocytes, reducing agents, and control(s).

14. An immunographic device for in vitro determining the vitamin status in a sample of a subject,
said device comprising a solid carrier or surface coated with anti-PLP-DE-antibodies or with nanoparticles comprising a moiety or tag which binds to a probe comprising at least one functionalized cofactor of PLP-dependent enzymes (PLP-DEs) (PLP probe).

15. The immunographic device of claim 14, wherein the immunographic device comprises a porous membrane operably connected to
(a) a portion or pad, where a processed sample is applied,
(b) a test portion or test line comprising said anti-PLP-DE-antibodies or said nanoparticles,
(c) a control portion or control line; and
(d) an absorbent portion/pad,
wherein the sample is whole blood or erythrocytes;
and/or wherein obtaining a processed sample comprises:
the erythrocytes or the harvested erythrocytes are lysed and thereby release the proteome including the PLP-dependent enzymes (PLP-DEs),
the lysate is treated with a probe comprising at least one functionalized cofactor of PLP-dependent enzymes (PLP-DEs) (PLP probe), and
a label for the PLP-probe is added to the treated lysate, and
the processed sample is obtained,
or wherein obtaining a processed sample comprises:
the erythrocytes or the harvested erythrocytes are treated with a probe comprising at least one functionalized cofactor of PLP-dependent enzymes (PLP-DEs) (PLP probe),
the treated erythrocytes are then lysed and thereby release the proteome including the PLP-dependent enzymes (PLP-DEs),
a label for the PLP-probe is added to the lysate, and
the processed sample is obtained.

16. The method of claim 1, wherein the vitamin is vitamin B6, B2, B12 and/or D3.

17. The method of claim 12, wherein the vitamin B6 deficiency and/or vitamin B6-associated disease is related to age, malnutrition and/or alcoholism; is in a dialysis patient; and/or is associated with inflammatory bowel disease.

18. The immunographic device of claim 14, wherein the vitamin is vitamin B6, B2, B12 and/or D3.

19. The immunographic device of claim 14, which is a lateral flow device.

20. The immunographic device of claim 14, which is a point of care device.
